(19) United States
(12) Patent Application Publication
(10) Pub. No.: US 2013/0071430 A1
(43) Pub. Date: Mar. 21, 2013

(54) MICRORNA-CONTROLLED RECOMBINANT VACCINIA VIRUS AND USE THEREOF

(71) Applicants: Takafumi Nakamura, Tokyo (JP); Mina Hikichi, Tokyo (JP); Hideaki Tahara, Tokyo (JP); Hisatoshi Shida, Hokkaido (JP); Minoru Kidokoro, Tokyo (JP)

(72) Inventors: Takafumi Nakamura, Tokyo (JP); Mina Hikichi, Tokyo (JP); Hideaki Tahara, Tokyo (JP); Hisatoshi Shida, Hokkaido (JP); Minoru Kidokoro, Tokyo (JP)

(73) Assignee: THE UNIVERSITY OF TOKYO, Tokyo (JP)

(21) Appl. No.: 13/639,784

(22) PCT Filed: Mar. 15, 2011
(86) PCT No.: PCT/JP2011/056693

§ 371 Date: Dec. 3, 2012

(30) Foreign Application Priority Data
Apr. 9, 2010 (JP) 2010-090662

(51) Int. Cl. C12N 7/00

(52) U.S. Cl. CPC C12N 7/00
USPC 424/204.1; 435/236; 435/320.1; 435/5; 424/277.1; 424/234.1; 424/269.1

(57) ABSTRACT

It is intended to provide a vaccinia virus that specifically proliferates in a cancer cell and destroys the cancer cell and to provide use of the virus in cancer treatment. The present invention provides a microRNA-controlled vaccinia virus, in which a target sequence of a microRNA less expressed in a cancer cell than in a normal cell is inserted in a 3′ untranslated region of B5R gene associated with viral proliferation in a vaccinia virus, wherein the microRNA-controlled vaccinia virus specifically proliferates in the cancer cell and has an oncolytic property that destroys the cancer cell.

### TECHNICAL FIELD

The present invention relates to a novel vaccinia virus and a virus vector comprising the same. Specifically, the present invention relates to a microRNA-controlled virus wherein a target sequence of a microRNA less expressed in cancer cells than in normal cells is inserted in the 3′ untranslated region of a gene associated with viral proliferation in a vaccinia virus. This microRNA-controlled vaccinia virus proliferates only in cancer cells with the gene associated with viral proliferation expressed in synchronization with gene expression regulation based on the microRNA-based control mechanism, thereby destroying the cancer cells. The present invention also relates to a vaccinia virus vector comprising the same.

### BACKGROUND ART

In recent years, various techniques have been developed on cancer virotherapy, which employs viruses in cancer treatment. Examples of the viruses used in such treatment include adenovirus and retrovirus as well as vaccinia virus.

A smallpox vaccine strain LC16m8 developed in Japan is an attenuated vaccinia virus strain in which the B5R gene associated with virus dissemination or virulence in host bodies has lost its function due to frameshift mutation. As a smallpox vaccine, this vaccine strain has been successfully inoculated into approximately 100,000 infants so far in Japan, in which no serious adverse reactions including death and immune responses equivalent to those observed with conventional vaccines were observed, and thus its high efficacy and safety has been proved (see So Hashizume, Clinical Virology, vol. 3, No. 3, 269, 1975). The B5R gene was completely deleted from this LC16m8 strain to develop a more genetically stable modified vaccine strain LC16m8Δ (see International Publication No. WO2005/054451).

In recent years, the vaccinia virus has also been used as a multivalent vaccine for infection (HIV or SARS) in the form of an expression vector containing a foreign gene on the basis of its properties such as a wide host range and high expression efficiency.

Meanwhile, the relation of microRNA (miRNA) to cancer has received attention in recent years. Reportedly, particular microRNAs are downregulated or upregulated in cancer cells (see, e.g., Steven M. Johnson et al., Cell 120: 635-647, 2005 and Carlo M. Croce et al., Nat. Rev. Genet. 10: 704-714, 2009).

### SUMMARY OF INVENTION

An object of the present invention is to provide a vaccinia virus that specifically proliferates in cancer cells and destroys the cancer cells and to use the virus in cancer treatment.

Preclinical research and clinical trials are currently being conducted actively wordwide on cancer virotherapy, which treats cancer using live viruses. The biggest key point of this virotherapy is how to eliminate the original virulence of viruses to normal tissues.

The present inventors have used a gene recombination technique based on the LC16m8Δ vaccine strain having established high safety to produce, with its high safety maintained, a recombinant vaccinia virus that specifically proliferates in cancer cells and destroys the cancer cells, and have consequently established cancer-specific virotherapy attacking only cancer tissues. Specifically, the B5R gene involved in the proliferation or virulence of vaccinia virus, together with, in its 3′ untranslated region, a target sequence of a microRNA less expressed in cancer cells than in normal cells, has been inserted into LC16m8Δ. As a result, it has been found that microRNA-based control represses the expression of the B5R gene in the vaccinia virus in normal cells to prevent the vaccinia virus from proliferating and damaging the normal cells, whereas the vaccinia virus efficiently proliferates in cancer cells with low microRNA expression without repression of the expression of the B5R gene, thereby specifically damaging only the cancer cells. Based on these findings, the present inventors have completed a method for treating cancer using a recombinant vaccinia virus whose proliferation is controlled by a microRNA.

Specifically, the present invention is as follows:

[1] A microRNA-controlled vaccinia virus, in which a target sequence of a microRNA less expressed in a cancer cell than in a normal cell is inserted in a 3′ untranslated region of B5R gene associated with viral proliferation in a vaccinia virus, wherein the microRNA-controlled vaccinia virus specifically proliferates in cancer cell and has an oncolytic property that specifically destroys the cancer cell.
[2] The microRNA-controlled vaccinia virus according to [1], wherein the microRNA expressed in the normal cell represses the expression of the B5R gene to reduce the proliferative capacity of the microRNA-controlled vaccinia virus in the normal cell.
[3] The microRNA-controlled vaccinia virus according to [1] or [2], wherein the B5R gene into which the microRNA target sequence is inserted in its 3′ untranslated region is introduced into an attenuated vaccinia virus lacking a portion or the whole of its B5R gene.
[4] The microRNA-controlled vaccinia virus according to [1] or [2], wherein the vaccinia virus is an LC16 strain or an LC 16 mO strain.
[5] The microRNA-controlled vaccinia virus according to [3], wherein the vaccinia virus is an LC16m8 strain lacking a portion of its B5R gene or an m8Δ strain lacking the whole of its B5R gene.
[6] The microRNA-controlled vaccinia virus according to any of [1] to [5], wherein the microRNA less expressed in a cancer cell than in a normal cell is selected from the group consisting of let-7a (SEQ ID NO: 1), let-7b (SEQ ID NO: 2), let-7c (SEQ ID NO: 3), let-7d (SEQ ID NO: 4), let-7e (SEQ ID NO: 5), let-7f (SEQ ID NO: 6), miR-9 (SEQ ID NO: 7), miR-15a (SEQ ID NO: 8), miR-16-1 (SEQ ID NO: 9), miR-21 (SEQ ID NO: 10), miR-20a (SEQ ID NO: 11), miR-26a (SEQ ID NO: 12), miR-27b (SEQ ID NO: 13), miR-29a (SEQ ID NO: 14), miR-29b (SEQ ID NO: 15), miR-29c (SEQ ID NO: 16), miR-30a (SEQ ID NO: 17), miR-30d (SEQ ID NO: 18), miR-32 (SEQ ID NO: 19), miR-33a (SEQ ID NO: 20), miR-34a (SEQ ID NO: 21), miR-92a (SEQ ID NO: 22), miR-95 (SEQ ID NO: 23), miR-101 (SEQ ID NO: 24), miR-122 (SEQ ID NO: 25), miR-124 (SEQ ID NO: 26), miR-125a (SEQ ID NO: 27), miR-125b (SEQ ID NO: 28), miR-126 (SEQ ID NO: 29), miR-127 (SEQ ID NO: 30), miR-128 (SEQ ID NO: 31), miR-133b (SEQ ID NO: 32), miR-139-5p (SEQ ID NO: 33), miR-140 (SEQ ID NO: 34), miR-141 (SEQ ID NO: 35), miR-142 (SEQ ID NO: 36), miR-143 (SEQ ID NO: 37), miR-144 (SEQ ID NO: 38), miR-145 (SEQ ID NO: 39), miR-155 (SEQ ID NO: 40), miR-181a (SEQ ID NO: 41), miR-181b (SEQ ID NO: 42), miR-181c (SEQ ID NO: 43), miR-192 (SEQ ID NO: 44), miR-195 (SEQ ID NO: 45), miR-198 (SEQ ID NO: 46), miR-199a (SEQ ID NO: 47), miR-199b-5p (SEQ ID NO: 48), miR-200a (SEQ ID NO: 49), miR-203 (SEQ ID NO: 50), miR-204 (SEQ ID NO: 51), miR-205 (SEQ ID NO: 52), miR-217 (SEQ ID NO: 53), miR-218 (SEQ ID NO: 54), miR-219-5p (SEQ ID NO: 55), miR-220a (SEQ ID NO: 56), miR-220b (SEQ ID NO: 57), miR-220c (SEQ ID NO: 58), miR-222 (SEQ ID NO: 59), miR-223 (SEQ ID NO: 60), miR-224 (SEQ ID NO: 61), miR-345 (SEQ ID NO: 62), and miR-375 (SEQ ID NO: 63).
[7] The microRNA-controlled vaccinia virus according to any of [1] to [6], wherein the microRNA-controlled vaccinia virus is deficient in one or more gene(s) whose loss of function resulting from deletion of the gene(s) is compensated for in the cancer cell, but is not compensated for in the normal cell.
[8] The microRNA-controlled vaccinia virus according to [7], wherein the microRNA-controlled vaccinia virus is deficient at least in a thymidine kinase gene.
[9] The microRNA-controlled vaccinia virus according to [8], wherein the microRNA-controlled vaccinia virus is further deficient in a hemagglutinin (HA) gene.
[10] The microRNA-controlled vaccinia virus according to [9], wherein the microRNA-controlled vaccinia virus is further deficient in an F fragment.
[11] The microRNA-controlled vaccinia virus according to [8], wherein the microRNA-controlled vaccinia virus is further deficient in a VGF gene.
[12] A pharmaceutical composition for cancer treatment, comprising a microRNA-controlled vaccinia virus according to any of [1] to [11].
[13] A microRNA-controlled vaccinia virus vector comprising a foreign DNA introduced in a microRNA-controlled vaccinia virus according to any of [1] to [12].
[14] The microRNA-controlled vaccinia virus vector according to [13], wherein the foreign DNA is a marker DNA, a therapeutic gene having cytotoxic effect or immunostimulating effect, or a DNA encoding a cancer, viral, bacterial, or protozoal antigen.
[15] A pharmaceutical composition for cancer treatment or for use as a vaccine against a cancer, a virus, a bacterium, or a protozoan, comprising a microRNA-controlled vaccinia virus vector according to [13] or [14].
[16] A method for evaluating the therapeutic effect of a microRNA-controlled vaccinia virus according to any of [1] to [11] on cancer in a cancer patient, comprising the steps of:
(i) contacting the microRNA-controlled vaccinia virus with a cancer cell and a normal cell collected from the cancer patient; and
(ii) assaying the proliferation of the microRNA-controlled vaccinia virus in the cancer cell and the normal cell wherein
the microRNA-controlled vaccinia virus is determined to have therapeutic effect on cancer when proliferating in the cancer cell and not proliferating in the normal cell.
[17] The method according to [16], wherein the microRNA-controlled vaccinia virus has a fusion gene of a B5R gene and a marker gene, and the therapeutic effect on cancer is evaluated on the basis of marker expression.

The proliferation of the microRNA-controlled vaccinia virus of the present invention is controlled depending on the expression level of a particular microRNA. The microRNA-controlled vaccinia virus fails to proliferate in cells with the increased expression of the particular microRNA because the expression of the B5R gene is repressed. By contrast, the expression of the B5R gene is induced in cells with the reduced expression of the microRNA so that the virus proliferates and damages the cells. Thus, a microRNA less expressed in a cancer cell than in a normal cell is selected as the particular microRNA. The resulting vaccinia virus efficiently proliferates in cancer cells and exerts potent antitumor effect. Thus, samples from individual cancer patients are examined for their microRNA expression in advance, and microRNAs can be analyzed in cancer tissues to select an appropriate microRNA. As a result, tailor-made drug development can be achieved, in which a microRNA-controlled vaccinia virus attacking only cancer cells can be selected.

In the present invention, a gene recombination technique based on, for example, the vaccinia virus vaccine strain LC16m8Δ having established high safety can be used to provide, with its high safety maintained, a microRNA-controlled recombinant virus that specifically proliferates in cancer cells and destroys the cancer cells.

Because of the wide host range and high expression efficiency of vaccinia virus, the microRNA-controlled recombinant virus of the present invention further functions as a vector containing an additional foreign gene. A microRNA-controlled recombinant vaccinia virus expressing luciferase or GFP allows convenient and rapid identification of cells infected therewith. In addition, the expression of a therapeutic gene having cytotoxic effect or immunostimulating effect also allows combined use thereof with other treatment methods.

The present specification incorporates the contents described in the specification and/or drawings of Japanese Patent Application No. 2010-090662, which serves as a basis of the priority of the present application.

### BRIEF DESCRIPTION OF THE DRAWINGS

FIG. 1 is a photograph showing the antitumor effect of an attenuated vaccinia virus on mouse models peritoneally inoculated with a human pancreatic cancer cell line BxPC-3.

FIG. 2 is a diagram showing the virulence of the attenuated vaccinia virus to mouse models peritoneally inoculated with a human pancreatic cancer cell line BxPC-3 (cross mark: dead). FIG. 2A shows results about Mock; FIG. 2B shows results about LC16mO; and FIG. 2C shows results about LC16m8Δ.

FIG. 3 is a diagram showing the genomic structure of a recombinant virus having a B5R gene insert.

FIG. 4 is a diagram showing the cell-killing effects of a recombinant virus lacking the B5R gene and the recombinant virus having a B5R gene insert on human cancer cells.

FIG. 5 is a diagram showing the new strategy of cancer-specific virotherapy development using the properties of cancer microRNA.

FIG. 6 is a diagram showing the relative expression level of let7a in human cancer cells.

FIG. 7 is a diagram showing the genomic structure of a let7a-controlled recombinant virus expressing GFP-tagged B5R.

FIG. 8 is a photograph showing the B5R expression and cytopathic effect of the let7a-controlled recombinant virus expressing GFP-tagged B5R in human cancer cells.

FIG. 9 is a diagram showing the proliferative capacity of the let7a-controlled recombinant virus expressing GFP-tagged B5R in human cancer cells.

FIG. 10 is a diagram showing the genomic structure of a let7a-controlled recombinant virus expressing two types of foreign genes.

FIG. 11 is a diagram showing the cell-killing effect of the let7a-controlled recombinant virus on cancer cells.

FIG. 12A is a photograph showing the biodistribution of the let7a-controlled recombinant virus in SCID mice.

FIG. 12B is a diagram showing results of numerically converting proliferating viruses in SCID mice.

FIG. 13A is a diagram showing the effect (tumor growth curve) of cancer virotherapy using a let7a-controlled recombinant virus on mouse models subcutaneously inoculated with human BxPC-3.

FIG. 13B is a diagram showing the effect (survival curve) of cancer virotherapy using the let7a-controlled recombinant virus on mouse models subcutaneously inoculated with human BxPC-3.

FIG. 14A is a diagram showing the effect (tumor growth curve) of cancer virotherapy using the let7a-controlled recombinant virus on mouse models subcutaneously inoculated with human lung cancer cell line A549.

FIG. 14B is a diagram showing the effect (survival curve) of cancer virotherapy using the let7a-controlled recombinant virus on mouse models subcutaneously inoculated with human lung cancer cell line A549.

FIG. 15 is a diagram showing the biodistribution and antitumor effect of the let7a-controlled recombinant virus in mouse models subcutaneously inoculated with human BxPC-3.

FIG. 16 is a photograph showing the biodistribution of the let7a-controlled recombinant virus in C57BL/6 mice.

FIG. 17 is a diagram showing the effect (survival curve) of cancer virotherapy using the let7a-controlled recombinant virus on mouse models intraperitoneally inoculated with human BxPC-3.

FIG. 18 is a diagram showing the genomic structure of a let7a-controlled recombinant virus lacking TK and expressing two types of foreign genes.

FIG. 19 is a diagram showing the effect (survival curve) of cancer virotherapy using the let7a-controlled recombinant virus lacking TK on mouse models intraperitoneally inoculated with human BxPC-3.

FIG. 20 is a photograph showing the biodistribution of the let7a-controlled recombinant virus lacking TK in mouse models intraperitoneally inoculated with human BxPC-3.

### DESCRIPTION OF EMBODIMENTS

Hereinafter, the present invention will be described in detail.

Examples of vaccinia virus strains for the production of the vaccinia virus of the present invention include, but not limited to, strains such as a Lister strain, LC16, LC16mO, and LC16m8 strains established from the Lister strain (So Hashizume, Clinical Virology, vol. 3, No. 3, 269, 1975), an NYBH strain, a Wyeth strain, and a Copenhagen strain. The LC16mO strain is a strain prepared via the LC16 strain from the Lister strain, while the LC16m8 strain is a strain further prepared from the LC16mO strain (Protein, Nucleic Acid and Enzyme, Vol. 48 No. 12 (2003), p. 1693-1700).

Preferably, the vaccinia virus used in the present invention has no virulence by attenuation, because of its established safety for administration to humans. Examples of such attenuated strains include strains lacking a portion or the whole of the B5R gene. The B5R gene encodes a protein present in the envelope of vaccinia virus. The B5R gene product is involved in viral infection and proliferation. The B5R gene product, which is located on the surface of infected cells or in the viral envelope, has the function of enhancing infection efficiency during the infection or dissemination of the virus in adjacent cells or other sites in the host body, and is also involved in the plaque size and host range of the virus. B5R gene deletion decreases a plaque size resulting from the infection of animal cells and also decreases a pock size. This deletion also reduces the proliferative capacity of the virus in the skin and reduces its cutaneous virulence. The vaccinia virus lacking a portion or the whole of its B5R gene has a small proliferative capacity in the skin without the normal functions of the B5R gene product and causes no adverse reaction even when administered to humans. Examples of the attenuated strain lacking the B5R gene include an m84 strain (also called LC16m8Δ strain), which has been established by the deletion of the whole B5R gene from the LC16m8 strain. Alternatively, an mOΔ strain (also called LCmOΔ strain) may be used, which has been established by the deletion of the whole B5R gene from the LC16mO strain. These attenuated vaccinia virus strains lacking a portion or the whole of the B5R gene are described in the pamphlet of International Publication No. WO2005/054451 and can be obtained on the basis of the description thereof. Whether or not a vaccinia virus lacks a portion or the whole of the B5R gene and loses B5R protein functions can be determined using, for example, the size of plaques formed by the infection of RK13 cells, a pock size, a viral proliferative capacity in Vero cells, or cutaneous virulence in rabbits as an index. Alternatively, the gene sequence of the vaccinia virus may be examined.

The vaccinia virus used in the present invention expresses the B5R gene in cancer cells and damages the cancer cells by the action of the B5R protein. Thus, the vaccinia virus used in the present invention must retain the complete B5R gene. In the case of using the attenuated vaccinia virus lacking the B5R gene and having established safety as described above, the complete B5R gene is newly introduced into the vaccinia virus lacking the B5R gene. In the case of using the vaccinia virus lacking a portion or the whole of the B5R gene, a B5R gene sequence containing a untranslated region, particularly, a 3′ untranslated region is inserted to the genome of the vaccinia virus, which can then be used as a material for the production of the vaccinia virus of the present invention. The insertion of the B5R gene to the vaccinia virus may be performed by any method and can be performed by, for example, a homologous recombination method known in the art. In this case, the position to which the B5R gene is inserted may be the original B5R gene position between the B4R and B6R genes or may be an arbitrary site on the genome of the vaccinia virus. Alternatively, a DNA construct of B5R gene containing a target sequence insert in its 3′ untranslated region may be prepared in advance and introduced to the vaccinia virus. The sequence of a portion comprising the B4R, B5R, and B6R gene sequences on the vaccinia virus genome is shown in SEQ ID NO: 87. The region from the 1780th a to the 2733rd a in SEQ ID NO: 87 represents ORF encoding the B5R protein. The 3′ untranslated region is located downstream of this stop codon.

The homologous recombination refers to a phenomenon that causes the mutual recombination between two DNA molecules via identical nucleotide sequences in a cell. This method is frequently used in the recombination of viruses having large genomic DNA, such as vaccinia virus. First, a plasmid comprising a B5R gene linked to the sequence of a targeted vaccinia virus gene site in a centrally divided form (this plasmid is referred to as a transfer vector) is constructed and introduced to vaccinia virus-infected cells to cause the replacement between identical sequence portions on the DNA of the virus rendered naked during viral replication and on the transfer vector so that the sandwiched B5R gene is incorporated into the viral genome. Examples of the cells that may be used in this procedure include cells infectible with vaccinia virus, such as BSC-1 cells, HTK-143 cells, Hep2 cells, MDCK cells, Vero cells, HeLa cells, CV1 cells, COS cells, RK13 cells, BHK-21 cells, and primary rabbit kidney cells. The introduction of the vector to the cells can be performed by a method known in the art, such as a calcium phosphate, cationic liposome, or electroporation method.

In most cases, microRNA (miRNA), a small RNA molecule consisting of 19 to 23 bases, inhibits the translation of messenger RNA (mRNA) of a particular gene or degrades the mRNA through its binding to a target site present in the 3′ untranslated region of the mRNA, thereby repressing protein expression. Since the target sequence (target site) comprises a sequence completely or partially complementary to the sequence of the miRNA, the miRNA controls the expression of the particular gene through its binding to the target sequence.

In the present invention, a miRNA less expressed in cancer cells than in normal cells is used. A target sequence of the miRNA less expressed in cancer cells than in normal cells is inserted to the 3′ untranslated region of the B5R gene in the vaccinia virus used. In normal cells, the miRNA binds to the target sequence, thereby repressing the expression of the B5R gene. Thus, the vaccinia virus does not exhibit virulence to the normal cells. By contrast, in cancer cells, which have the low expression of the miRNA, the miRNA does not bind to the target sequence. As a result, the B5R gene is expressed without being repressed, to produce the B5R protein. This B5R protein normally functions in the cancer cells so that the vaccinia virus specifically proliferates in the cancer cells and has an oncolytic property that destroys and damages the cancer cells. Specifically, the vaccinia virus of the present invention is oncolytic in a cancer cell-specific manner.

The miRNA less expressed in cancer cells than in normal cells encompasses, without limitations, all of currently known miRNAs and miRNAs that may be found in the future. The sequence or origin of each miRNA can be confirmed by the search of a miRNA-related database, for example, the miRBase sequence database (http://microrna.sanger.ac.uk/sequences/index.shtml). Alternatively, a miRNA downregulated in cancer cells, as described in, for example, The Journal of Experimental Medicine, Vol. 27, No. 8 (May issue) 2009, p. 1188-1193 and p. 1218-1222; Yong Sun Lee and Anindya Dutta, “MicroRNAs in Cancer”, Annu. Rev. Pathol. Mech, DIs, 2009. 4: 199-227; and Carlo M. Croce, NATURE REVIEWS, Volume 10, October 2009, 704-714, can be selected.

Examples of the human-derived miRNAs less expressed in cancer cells than in normal cells include those shown below together with miRNA names and the sequence of each mature miRNA (the following sequences are indicated in the direction of 5′→3′):

let-7a
(SEQ ID NO: 1)
UGAGGUAGUAGGUUGUAUAGUU
let-7b
(SEQ ID NO: 2)
UGAGGUAGUAGGUUGUGUGGUU
let-7c
(SEQ ID NO: 3)
UGAGGUAGUAGGUUGUAUGGUU
let-7d
(SEQ ID NO: 4)
AGAGGUAGUAGGUUGCAUAGUU
let-7e
(SEQ ID NO: 5)
UGAGGUAGGAGGUUGUAUAGUU
let-7f
(SEQ ID NO: 6)
UGAGGUAGUAGAUUGUAUAGUU
miR-9
(SEQ ID NO: 7)
UCUUUGGUUAUCUAGCUGUAUGA
miR-15a
(SEQ ID NO: 8)
UAGCAGCACAUAAUGGUUUGUG
miR-16-1
(SEQ ID NO: 9)
UAGCAGCACGUAAAUAUUGGCG
miR-21
(SEQ ID NO: 10)
UAGCUUAUCAGACUGAUGUUGA
miR-20a
(SEQ ID NO: 11)
UAAAGUGCUUAUAGUGCAGGUAG
miR-26a
(SEQ ID NO: 12)
UUCAAGUAAUCCAGGAUAGGCU
miR-27b
(SEQ ID NO: 13)
UUCACAGUGGCUAAGUUCUGC
miR-29a
(SEQ ID NO: 14)
UAGCACCAUCUGAAAUCGGUUA
miR-29b
(SEQ ID NO: 15)
UAGCACCAUUUGAAAUCAGUGUU
miR-29c
(SEQ ID NO: 16)
UAGCACCAUUUGAAAUCGGUUA
miR-30a
(SEQ ID NO: 17)
UGUAAACAUCCUCGACUGGAAG
miR-30d
(SEQ ID NO: 18)
UGUAAACAUCCCCGACUGGAAG
miR-32
(SEQ ID NO: 19)
UAUUGCACAUUACUAAGUUGCA
miR-33a
(SEQ ID NO: 20)
GUGCAUUGUAGUUGCAUUGCA
miR-34a
(SEQ ID NO: 21)
UGGCAGUGUCUUAGCUGGUUGU
miR-92a
(SEQ ID NO: 22)
UAUUGCACUUGUCCCGGCCUGU
miR-95
(SEQ ID NO: 23)
UUCAACGGGUAUUUAUUGAGCA
miR-101
(SEQ ID NO: 24)
UACAGUACUGUGAUAACUGAA
miR-122
(SEQ ID NO: 25)
UGGAGUGUGACAAUGGUGUUUG
miR-124
(SEQ ID NO: 26)
UAAGGCACGCGGUGAAUGCC
miR-125a
(SEQ ID NO: 27)
UCCCUGAGACCCUUUAACCUGUGA
miR-125b
(SEQ ID NO: 28)
UCCCUGAGACCCUAACUUGUGA
miR-126
(SEQ ID NO: 29)
UCGUACCGUGAGUAAUAAUGCG
miR-127
(SEQ ID NO: 30)
CUGAAGCUCAGAGGGCUCUGAU
miR-128
(SEQ ID NO: 31)
UCACAGUGAACCGGUCUCUUU
miR-133b
(SEQ ID NO: 32)
UUUGGUCCCCUUCAACCAGCUA
miR-139-5p
(SEQ ID NO: 33)
UCUACAGUGCACGUGUCUCCAG
miR-140
(SEQ ID NO: 34)
CAGUGGUUUUACCCUAUGGUAG
miR-141
(SEQ ID NO: 35)
UAACACUGUCUGGUAAAGAUGG
miR-142
(SEQ ID NO: 36)
CAUAAAGUAGAAAGCACUACU
miR-143
(SEQ ID NO: 37)
UGAGAUGAAGCACUGUAGCUC
miR-144
(SEQ ID NO: 38)
UACAGUAUAGAUGAUGUACU
miR-145
(SEQ ID NO: 39)
GUCCAGUUUUCCCAGGAAUCCCU
miR-155
(SEQ ID NO: 40)
UUAAUGCUAAUCGUGAUAGGGGU
miR-181a
(SEQ ID NO: 41)
AACAUUCAACGCUGUCGGUGAGU
miR-181b
(SEQ ID NO: 42)
AACAUUCAUUGCUGUCGGUGGGU
miR-181c
(SEQ ID NO: 43)
AACAUUCAACCUGUCGGUGAGU
miR-192
(SEQ ID NO: 44)
CUGACCUAUGAAUUGACAGCC
miR-195
(SEQ ID NO: 45)
UAGCAGCACAGAAAUAUUGGC
miR-198
(SEQ ID NO: 46)
GGUCCAGAGGGGAGAUAGGUUC
miR-199a
(SEQ ID NO: 47)
CCCAGUGUUCAGACUACCUGUUC
miR-199b-5p
(SEQ ID NO: 48)
CCCAGUGUUUAGACUAUCUGUUC
miR-200a
(SEQ ID NO: 49)
UAACACUGUCUGGUAACGAUGU
miR-203
(SEQ ID NO: 50)
GUGAAAUGUUUAGGACCACUAG
miR-204
(SEQ ID NO: 51)
UUCCCUUUGUCAUCCUAUGCCU
miR-205
(SEQ ID NO: 52)
UCCUUCAUUCCACCGGAGUCUG
miR-217
(SEQ ID NO: 53)
UACUGCAUCAGGAACUGAUUGGA
miR-218
(SEQ ID NO: 54)
UUGUGCUUGAUCUAACCAUGU
miR-219-5p
(SEQ ID NO: 55)
UGAUUGUCCAAACGCAAUUCU
miR-220a
(SEQ ID NO: 56)
CCACACCGUAUCUGACACUUU
miR-220b
(SEQ ID NO: 57)
CCACCACCGUGUCUGACACUU
miR-220c
(SEQ ID NO: 58)
ACACAGGGCUGUUGUGAAGACU
miR-222
(SEQ ID NO: 59)
AGCUACAUCUGGCUACUGGGU
miR-223
(SEQ ID NO: 60)
UGUCAGUUUGUCAAAUACCCCA
miR-224
(SEQ ID NO: 61)
CAAGUCACUAGUGGUUCCGUU
miR-345
(SEQ ID NO: 62)
GCUGACUCCUAGUCCAGGGCUC
miR-375
(SEQ ID NO: 63)
UUUGUUCGUUCGGCUCGCGUGA

The less expressed miRNAs differ depending on the type of cancer. For example, miR-128 and miR-181 in brain tumor, let-7, miR-15a, miR-16, miR-125a, miR-125b, miR-127, miR-145, and miR-204 in breast cancer, let-7, miR-9, miR-26a, miR-27b, miR-29b, miR-32, miR-33, miR-30a, miR-95, miR-101, miR-124, miR-125a, miR-126, miR-140, miR-143, miR-145, miR-198, miR-192, miR-199b, miR-218, miR-219, miR-220, miR-224, miR-203, and miR-205 in lung cancer, miR-203 and miR-205 in esophagus cancer, let-7 in gastric cancer, let-7, miR-34, miR-127, miR-133b, miR-143, and miR-145 in colorectal cancer, let-7, miR-101, miR-122, miR-125a, miR-195, miR-199a, and miR-200a in hepatocellular carcinoma, miR-139, miR-142, miR-345, and miR-375 in pancreatic cancer, miR-15a, miR-16, miR-143, miR-145, and miR-218 in prostatic cancer, miR-143 and miR-145 in uterine cervix cancer, or miR-15a, miR-16, miR-143, miR-145, miR-192, and miR-220 in B-CCL (B-cell chronic lymphocytic leukemia) are less expressed than in normal cells. Thus, in the case of using the vaccinia virus of the present invention in the treatment of a particular cancer type, a miRNA whose expression is specifically or particularly downregulated in the particular cancer type may be used. However, each miRNA is not necessarily downregulated only in the particular cancer, but is downregulated in cells of various cancer types to a greater or lesser extent. Thus, any miRNA can be used in cancer treatment, regardless of cancer types.

Among the miRNAs described above, let-7a is preferable because this miRNA is low expressed in clinical samples of lung cancer, pancreatic cancer, melanoma, or the like and contributes to the establishment of novel methods for treating intractable malignant tumors highly resistant to existing treatment methods. Also, a miRNA such as miR-15, miR-16, miR-143, or miR-145 much less expressed in cancer cells than in normal cells can be used preferably.

The miRNA binds to mRNA comprising, in its 3′ untranslated region, a sequence partially or completely complementary to the miRNA sequence, thereby repressing the expression of the particular gene by the inhibition of the translation of the mRNA or by the degradation of the mRNA. Thus, in the present invention, a target sequence of the miRNA is inserted as a miRNA-binding site to the 3′ untranslated region (3′-UTR) of the B5R gene in the vaccinia virus. The insertion position is not limited and may be any site within the 3′ terminal region including both ends of the 3′ untranslated region. For example, the target sequence can be inserted immediately downstream of the stop codon of B5R protein-encoding ORF. The region from the 1780th a to the 2733rd a in the nucleotide sequence represented by SEQ ID NO: 87 is the coding nucleotide sequence of B5R protein ORF. The miRNA target sequence can be inserted downstream of the stop codon of this ORF.

The target sequence comprises a sequence complementary to the partial or whole sequence of the miRNA. Its base length is 7 to 25 bases long, preferably 15 to 25 bases long, more preferably 19 to 23 bases long. Preferably, the target sequence consists of a sequence completely complementary to the sequence of its corresponding miRNA. However, the target sequence may have one or more, for example, 1 to 3, 1 or 2, or 1 mismatch(s) as long as it is capable of hybridizing to the miRNA. In this case, the hybridization conditions are in vivo conditions when the vaccinia virus of the present invention is administered to living bodies for pharmaceutical use. Alternatively, moderately or highly stringent conditions are adopted when the vaccinia virus of the present invention is used in vitro as a reagent. Examples of such conditions include conditions involving hybridization at 50° C. to 70° C. for 12 to 16 hours in 400 mM NaCI, 40 mM PIPES (pH 6.4), and 1 mM EDTA. Alternatively, the target sequence has 95% or higher, preferably 96, 97, 98, or 99% or higher sequence identity to the sequence completely complementary to the miRNA sequence of the present invention in terms of a numeric value calculated using default parameters in a homology search program known by those skilled in the art, such as BLAST [J. Mol. Biol., 215, 403-410 (1990)] or FASTA [Methods. Enzymol., 183, 63-98 (1990)]. Also, one or more, for example, 1 to 3, 1 or 2, or 1 base(s) may be added to one or both of the ends of this completely complementary sequence.

Since the miRNA binds to mRNA transcribed from DNA, the target sequence to be inserted in the 3′ untranslated region of the B5R gene in the vaccinia virus is a DNA sequence containing thymine (T) in a reverse complementary relationship with the miRNA sequence. Thus, this DNA sequence in a reverse complementary relationship can be inserted to the 3′ untranslated region of the B5R gene in the vaccinia virus. In the case of using, for example, let-7a miRNA having the sequence 5′-UGAGGUAGUAGGUUGUAUAGUU-3′ (SEQ ID NO: 1), the target sequence 5′-AACTATACAACCTACTACCTCA-3′ (SEQ ID NO: 64) can be inserted to the 3′ untranslated region of the B5R gene in the vaccinia virus. Those skilled in the art can appropriately design or determine the sequence of the binding site to be inserted to the 3′ untranslated region of the B5R gene in the vaccinia virus.

At least one target sequence may be present in the 3′ untranslated region of the B5R gene in the vaccinia virus. Alternatively, a plurality of repeat sequences of each target sequence may be present therein. In the case of the plurality of repeat sequences, the number of repeats is 2 to 20, preferably 2 to 10, more preferably 2 to 5, further preferably 2 to 4. Not only a target sequence for one miRNA but a plurality of target sequences for different miRNAs may be inserted thereto. In this case, a spacer sequence may be inserted to between these miRNA target sequences. The length and bases of the spacer sequence are not limited, and, for example, a nucleotide sequence of 3 to 10 bases, preferably 3 to 5 bases long, can be inserted to between the target sequences.

The present invention utilizes a miRNA less expressed in cancer cells than in normal cells. The degree of downregulation of the particular miRNA may differ among patients. In addition, the degree of downregulation of the particular miRNA may differ among cancer types. A miRNA particularly downregulated in cancer cells can be selected for each patient in advance, or a miRNA specifically or particularly downregulated in a particular cancer type can be selected, to achieve more specifically effective treatment on a patient or cancer type basis.

The vaccinia virus of the present invention comprising the miRNA target sequence as a binding site in the 3′ untranslated region of the B5R gene is referred to as a miRNA-controlled vaccinia virus or a miRNA-controlled proliferation-type vaccinia virus.

The miRNA-controlled vaccinia virus of the present invention can be used for cancer treatment. Specifically, the present invention encompasses a pharmaceutical composition for cancer treatment, comprising the miRNA-controlled vaccinia virus.

The cancer targeted by the miRNA-controlled vaccinia virus of the present invention is not limited. The miRNA-controlled vaccinia virus can target every cancer type including, for example, skin cancer, gastric cancer, lung cancer, liver cancer, colon cancer, pancreatic cancer, anal/rectal cancer, esophagus cancer, uterine cancer, breast cancer, bladder cancer, prostatic cancer, esophagus cancer, ovarian cancer, brain/neural tumor, lymphoma/leukemia, osteoma/osteosarcoma, leiomyoma, and rhabdomyoma according to classification based on affected organs.

The pharmaceutical composition for cancer treatment, comprising the miRNA-controlled vaccinia virus of the present invention comprises a pharmaceutically effective amount of the vaccinia virus vaccine of the present invention as an active ingredient and may be in the form of a steric aqueous or nonaqueous solution, suspension, or emulsion. The pharmaceutical composition may further contain pharmaceutically acceptable diluents, aids, vehicles, etc., such as salts, buffers, and adjuvants. Its administration is achieved through various parenteral routes, for example, hypodermic, intravenous, intradermal, intramuscular, intraperitoneal, intranasal, and endermic routes. The effective dose can be determined appropriately according to the age, sex, health, and body weight, etc. of a test subject. For example, the dose in human adult is, but not limited to, approximately 10² to 10¹⁰ plaque forming unit (pfu), preferably 10⁵ to 10⁶ plaque forming unit (pfu), per administration.

The miRNA-controlled vaccinia virus of the present invention may further comprise a foreign gene (foreign DNA or foreign polynucleotide). Examples of the foreign gene (foreign DNA or foreign polynucleotide) include a marker gene and a therapeutic gene encoding a product having cytotoxic or immunostimulating effect and further include DNAs encoding cancer, viral, bacterial, or protozoal protein antigens. Examples of the marker gene, also called reporter gene, include luciferase (LUC) gene, genes of fluorescent proteins such as green fluorescent protein (GFP) and red fluorescent protein (DsRed), β-glucuronidase (GUS) gene, chloramphenicol acetyltransferase (CAT) gene, and (β-galactosidase (LacZ) gene. In the present invention, the miRNA-controlled vaccinia virus comprising any of these foreign genes can also be referred to as a miRNA-controlled vaccinia virus vector.

A miRNA-controlled vaccinia virus having an insert of any of these marker genes under the control of a promoter of the B5R gene, i.e., an insert of a fusion gene of the B5R gene and a marker gene, can be used for the evaluation of miRNA-based regulation. Specifically, a target sequence of a particular miRNA is inserted to the predetermined site of the vaccinia virus, with which particular cancer cells are then infected. In the case of using a miRNA effective for the control of vaccinia virus proliferation, i.e., using a miRNA low expressed in cancer cells, the marker gene is expressed, together with the B5R gene, in the cancer cells to produce the marker gene product such as fluorescent protein into the cells. The marker can be assayed to evaluate the efficacy of the miRNA used. The present invention encompasses a system and a method for evaluating a miRNA using a miRNA-controlled vaccinia virus comprising a marker gene. In order to select, for example, a miRNA suitable for a particular cancer patient, cancer cells and normal cells are collected from the patient and are both infected with miRNA-controlled vaccinia viruses respectively comprising target sequences of various miRNAs. As a result, a miRNA that causes the vaccinia virus to proliferate in the cancer cells and not to proliferate in the normal cells can be selected.

The therapeutic gene refers to a gene that may be used in the treatment of a particular disease such as cancer or infection. Examples thereof include tumor suppressor genes such as p53 and Rb, and genes encoding biologically active substances such as interleukin 1 (IL-1), IL-2, IL-3, IL-4, IL-5, IL-6, IL-7, IL-8, IL-9, IL-10, IL-11, IL-12, IL-13, IL-14, IL-15, α-interferon, β-interferon, γ-interferon, angiostatin, thrombospondin, endostatin, METH-1, METH-2, GM-CSF, G-CSF, M-CSF, and tumor necrosis factors.

A DNA encoding, for example, a viral, bacterial, protozoal or cancer antigen may be introduced as a foreign gene (foreign DNA) thereto. The resulting vaccinia virus vector containing the foreign gene can be used as a vaccine against various viruses, bacteria, protozoans and cancers. For example, a gene encoding a protective antigen (neutralizing antigen) against human immunodeficiency virus, hepatitis virus, herpes virus, mycobacteria, malaria parasite, or severe acute respiratory syndrome (SARS) virus, or a cancer antigen can be introduced thereto.

These foreign genes can be introduced using, for example, a homologous recombination approach. The homologous recombination may be performed by the method described above. For example, a foreign gene to be introduced is linked into the DNA sequence of the desired site to prepare a plasmid (transfer vector), which can then be introduced into vaccinia virus-infected cells. For example, pSFJ1-10, pSFJ2-16, pMM4, pGS20, pSC11, pMJ601, p2001, pBCB01-3,06, pTKgpt-F1-3s, pTM1, pTM3, pPR34,35, pgpt-ATA18-2, or pHES1-3 can be used as the transfer vector. The region to which the foreign gene is introduced is preferably within a gene that is not essential for the life cycle of the vaccinia virus. Alternatively, the foreign gene may be inserted to, for example, a gene or a particular region whose loss of function attributed to the inserted foreign gene can be compensated for in cancer cells by virtue of abundant enzymes or the like of the cancer cells, but is not compensated for in normal cells. In this case, the miRNA-controlled vaccinia virus of the present invention can proliferate in cancer cells and destroys and damages the cancer cells by the action of the miRNA, whereas the miRNA-controlled vaccinia virus of the present invention fails to proliferate in normal cells and thus, neither destroys nor damages the normal cells. Examples of such genes include: hemagglutinin (HA) gene; thymidine kinase (TK) gene; F fragment; F3 gene; VGF gene (U.S. Patent Application Publication No. 2003/0031681); hemorrhagic region or type A inclusion body region (U.S. Pat. No. 6,596,279); Hind III F, F13L, or Hind III M region (U.S. Pat. No. 6,548,068); A33R, A34R, or A36R gene (Katz et al., J. Virology 77: 12266-12275 (2003)); SalF7L gene (Moore et al., EMBO J. 1992 11: 1973-1980); N1L gene (Kotwal et al., Virology 1989 171: 579-58); Ml gene (Child et al., Virology. 1990 174: 625-629); HR, HindIII-MK, HindIII-MKF, HindIII-CNM, RR, or BamF region (Lee et al., J. Virol. 1992 66: 2617-2630); and C21L gene (Isaacs et al., Proc Natl Acad Sci USA. 1992 89: 628-632). Among these genes, TK gene, HA gene, F fragment, or VGF gene is preferable. For example, the loss of function of the TK gene reduces the proliferative capacity of the vaccinia virus in normal cells. By contrast, the loss of function of the TK gene does not reduce the proliferative capacity thereof in cancer cells, which are rich in enzymes compensating for the functions of this gene. The reduced proliferative capacity in normal cells means reduced virulence to the normal cells, i.e., improves safety for application to living bodies. Cells infectible with vaccinia virus, such as Vero cells, HeLa cells, CV1 cells, COS cells, RK13 cells, BHK-21 cells, primary rabbit kidney cells, BSC-1 cells, HTK-143 cells, Hep2 cells, or MDCK cells, may be used as the cells to be infected with the vaccinia virus.

Also, the functions of the gene whose loss of function can be compensated for by virtue of abundant enzymes or the like of the cancer cells, but is not compensated for in normal cells may be deleted in the miRNA-controlled vaccinia virus of the present invention. Such a miRNA-controlled vaccinia virus in which the normal functions of the gene have been deleted proliferates in cancer cells and destroys and damages the cancer cells by the action of the miRNA, whereas the miRNA-controlled vaccinia virus fails to proliferate in normal cells and thus, neither destroys nor damages the normal cells. In this context, the deletion of the normal functions of the gene, also called deficiency in the gene, means that the gene is not expressed or, even if it is expressed, the expressed protein does not retain its normal functions. For the deletion of the normal functions of the gene, the foreign gene may be inserted in the gene, as described above, or the gene may be deleted partially or completely. The insertion of the foreign gene or the deletion of the gene can be performed by, for example, homologous recombination. Examples of the gene whose loss of function can be compensated for by virtue of abundant enzymes or the like of the cancer cells, but is not compensated for in normal cells include the genes exemplified above. Among them, TK gene, HA gene, F fragment, or VGF gene is preferable. One or more of these genes can be deleted. Particularly, deficiency in TK gene is preferable because this deficiency represses viral proliferation in normal tissues, resulting in the increased therapeutic index of the microRNA-controlled proliferation-type vaccinia virus. The microRNA-controlled vaccinia virus of the present invention may be deficient in HA gene and F fragment in addition to the TK gene or may be deficient in VGF gene in addition to the TK gene.

For the introduction of the foreign gene, preferably, an appropriate promoter is operably linked upstream of the foreign gene. Examples of the promoter that may be used include, but not limited to, PSFJ1-10 or PSFJ2-16 described above, p7.5K promoter, p11K promoter, T7.10 promoter, CPX promoter, HF promoter, H6 promoter, and T7 hybrid promoter. The introduction of the foreign gene to the vaccinia virus vector of the present invention can be performed by a method known in the art for constructing recombinant vaccinia virus vectors and can be performed according to the description of for example, The Journal of Experimental Medicine, suppl., The Protocol Series: Analytical and Experimental Methods for Gene Transfer & Expression, ed., by Izumi Saito, et al., Yodosha Co., Ltd. (issued on Sep. 1, 1997), DNA Cloning 4: A Practical Approach Mammalian Systems, ed., by D. M. Glover et al. (translation supervisor: Ikunoshin Kato), the 2nd edition, Takara Bio Inc., or EMBO Journal, (1987, Vol. 6 p. 3379-3384).

The present invention will be described specifically with reference to Examples below. However, the present invention is not intended to be limited to these Examples.

Next, the present invention will be described more specifically with reference to Examples.

### Reference Example 1

### Anticancer Effect (Oncolytic Effect) and Safety of Attenuated Vaccinia Virus

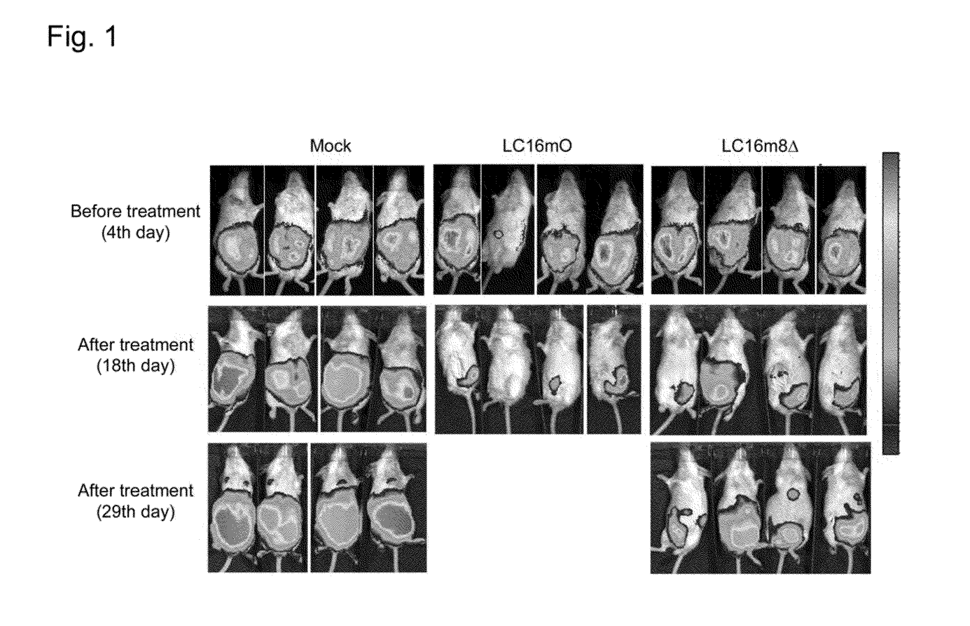

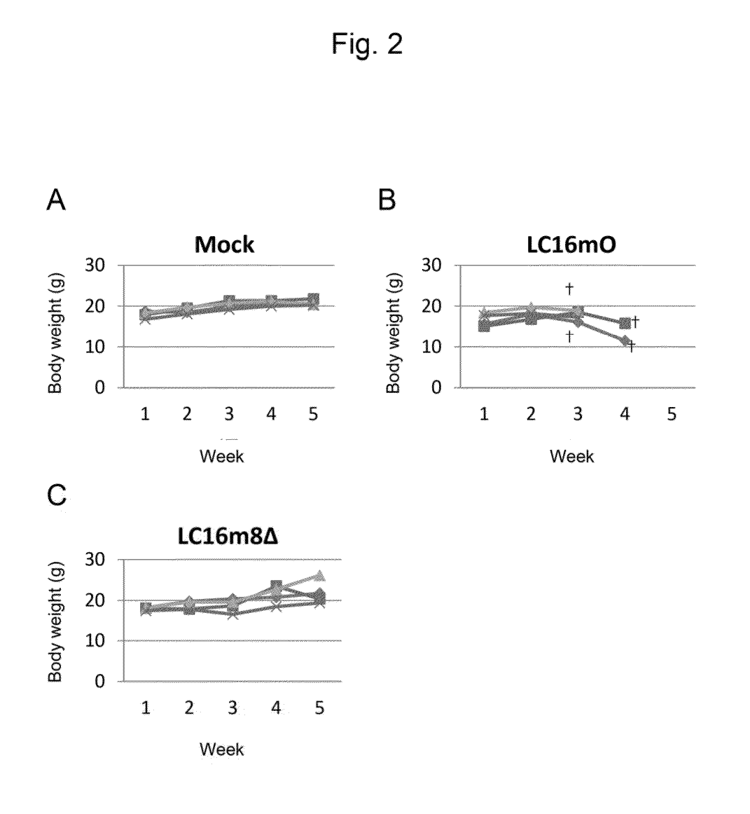

Human pancreatic cancer BxPC-3Luc cells (5×10⁶ cells) constitutively expressing luciferase were intraperitoneally administered to each SCID mouse to prepare an peritoneally inoculated mouse model. On the 4th day from the administration of the BxPC-3Luc cells, a luciferin solution (15 mg/mL in PBS) was intraperitoneally administered in an amount of 10 μL/g to the peritoneally inoculated mouse model. Luminescence derived from luciferase expressed in cancer cells was noninvasively monitored using IVIS™ imaging system (Xenogen Corp.) (FIG. 1; Before treatment). Next, on the 7th day from the administration of the BxPC-3Luc cells, 10⁷ plaque forming unit (pfu) of an LC16mO strain (intermediate strain obtained during the separation of an LC16m8 strain from the Lister strain; characteristic properties: attenuated virulence to the central nervous system) or LC16m8Δ (recombinant virus having the improved genetic stability of the LC16m8 strain and exhibiting properties similar to the C16 m8 strain historically used as a vaccine in humans without causing adverse reactions) was intraperitoneally administered to the peritoneally inoculated mouse model. In order to determine the anticancer effect of each virus, the cancer cells in the body of each mouse were monitored as described above on the 18th and 29th days from the administration of the BxPC-3Luc cells (FIG. 1; After treatment). As a result, the growth of the transplanted cancer cells over time was confirmed in a mock control group (Mock) inoculated with no virus. By contrast, potent antitumor effect was shown in the LC 16 mO-administered group. Temporal antitumor effect was shown in the LC16m8Δ-administered group, in which the regrowth of the transplanted cancer cells was however confirmed on the 29th day. At the same time therewith, each virus was evaluated for its adverse reaction in treatment on the basis of change in the body weights of the virus-administered mice. As a result, the LC16mO-administered mice all died in the period from the 21st to 28th days due to rapid weight loss with pocks developed by systemic viral proliferation. By contrast, no adverse reaction was seen in the LC16m8Δ-administered group, in totally the same way as in the mock control group (FIG. 2). These results suggested that the anticancer effect of LC16m8Δ itself was insufficient for completely killing cancer cells, though it could be a very highly safe oncolytic virus.

### Reference Example 2

### B5R Expression and Proliferative Capacity of Attenuated Vaccinia Virus in Human Tumor Cells

The gene region from B4R through B5R to B6R was amplified with the genomic DNA of the LC16mO strain as a template using two primers 5′-TCGGAAGCAGTCGCAAACAAC-3′ (SEQ ID NO: 65) and 5′-ATACCATCGTCGTTAAAAGCGC-3′ (SEQ ID NO: 66) and cloned into a TA vector pCRII (Invitrogen Corp.) to construct pB5R.

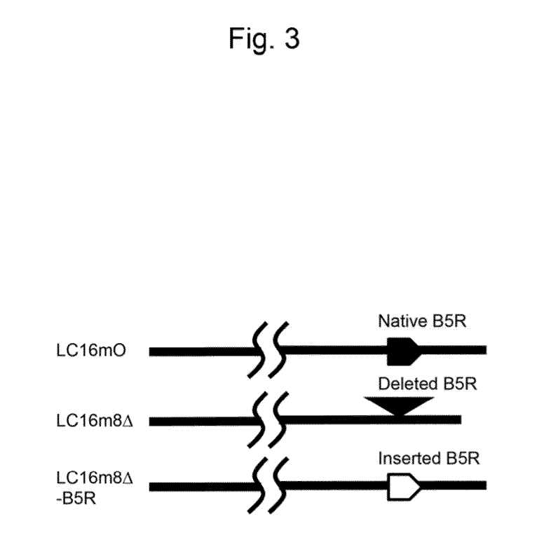

In order to recover a recombinant virus LC16m8Δ-B5R (FIG. 3), RK13 cells cultured until 80% confluence in a 6-well dish were infected with the vaccinia virus (LC16m8Δ) at MOI=0.02 to 0.1. After viral adsorption at room temperature for 1 hour, the transfer vector plasmid DNA (pB5R) mixed with FuGENE HD (Roche) was incorporated to cells by addition according to the manual and cultured at 37° C. for 2 to 5 days. The cells were frozen and thawed, then sonicated, and inoculated at an appropriate dilution ratio to the substantially confluent RK13 cells. An Eagle MEM medium containing 5% FBS and 0.8% methylcellulose was added thereto, and the cells were cultured at 37° C. for 2 to 5 days. The medium was removed, and large plaques were scraped off using the edge of a tip and suspended in an Opti-MEM medium (Invitrogen Corp.). This procedure, was further repeated three or more times using RK13 cells to purify the plaques. The suspension of the plaques collected after the plaque purification was sonicated, and a 200 μL aliquot thereof was then centrifuged at 15,000 rpm for 30 minutes. To the precipitates, 50 pt of sterile distilled water or 10 mM Tris-HCl (pH 7.5) was added. After 30-second sonication, the solution was heated at 95° C. for 10 minutes to extract genomic DNA, which was in turn subjected to screening by PCR. The PCR was performed using two primers 5′-cgtataatacgttggtctat-3′ (SEQ ID NO: 67) and 5′-gatcgtgccaatagtagtta-3′ (SEQ ID NO: 68), and the PCR product of the predetermined size in clones was detected. The nucleotide sequence of the PCR product was confirmed by direct sequencing. Viral clones without problems in the nucleotide sequence were selected and cultured in large amounts in RK13 cells. Then, the virus titer was determined in the RK13 cells for the subsequent experiment.

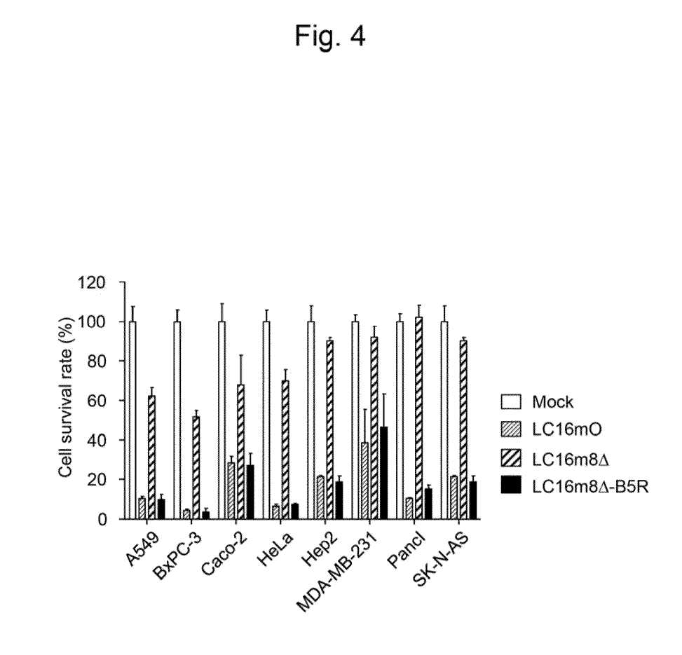

In order to study the oncolytic effect of each virus having the viral genome shown in FIG. 3, each human cancer cell line (lung cancer A549 cells, pancreatic cancer BxPC-3 and Panel cells, colon cancer Caco-2 cells, uterine cervix cancer HeLa cells, pharyngeal cancer HEp-2 cells, breast cancer MDA-MB-231 cells, and neuroblastoma SK-N-AS cells) cultured in a 96-well dish was infected with the virus at MOI=0.5 and cultured at 37° C. for 5 days. Then, the number of live cells was counted using CellTiter 96(R) AQueous One Solution Cell Proliferation Assay (Promega Corp.) according to the manual (FIG. 4). As a result, the B5R-expressing LC16mO strain and the recombinant LC16m8Δ-B5R exhibited equivalent oncolytic effect on all the cancer cells and killed approximately 60 to 95% cells with the number of live cells in the mock control group defined as 100%. By contrast, LC16m8Δ with no B5R expression exhibited oncolytic effect on some of the cell lines, but killed only 0 to 50% cells. These results demonstrated that the oncolytic effect of vaccinia virus that killed the infected tumor cells during its proliferation was drastically potentiated by B5R expression.

### Example 1

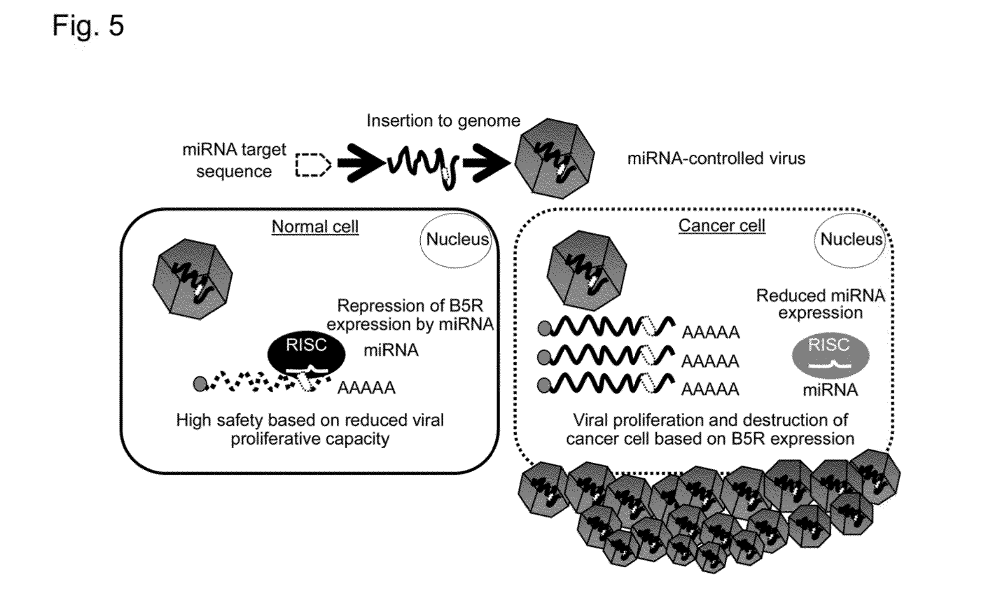

### Construction of MicroRNA-Controlled Proliferation-Type Vaccinia Virus (FIG. 5) Specifically Destroying Only Cancer Cells

In order to insert the sequences of NheI and AgeI restriction enzymes to B5R gene 3′ UTR, two types of DNA fragments were amplified with the pB5R plasmid as a template using each prime pair 5′-CAAACTCTCGAAAGACGT-3′ (SEQ ID NO: 69) and 5′-gcaccggtgctagcTTACGGTAGCAATTTATGGAA-3′ (SEQ ID NO: 70) or 5′-ccgctagcaccggtATATAAATCCGTTAAAATAATTAAT-3′ (SEQ ID NO: 71) and 5′-CAGGAAACAGCTATGAC-3′ (M13 reverse primer (SEQ ID NO: 72)). The PCR product of the former fragment was cleaved with restriction enzymes HpaI and NheI while the PCR product of the latter fragment was cleaved with restriction enzymes NheI and HindII. These two types of DNA fragments were cloned into pB5R cleaved with restriction enzymes HpaI and HindII to construct pTN-B5R. B5R with the stop codon removed was amplified with the pTN-B5R plasmid as a template using two primers 5′-caaaatattttcgttgcgaaga-3′ (SEQ ID NO: 73) and 5′-CACCATGGGTAGCAATTTATGGAACT-3′ (SEQ ID NO: 74). Also, EGFP (green fluorescent protein) gene with the sequence of NheI restriction enzyme added was amplified with a pEGFP-N1 (Clontech Laboratories, Inc.) plasmid as a template using two primers 5′-GCGGCCGGACCGGCCACCATGGTGAGCAAGGGCGA-3′ (SEQ ID NO: 75) and 5′-gcgctagcTTACTTGTACAGCTCGTCCA-3′ (SEQ ID NO: 76). The PCR product of the former gene was cleaved with restriction enzymes HpaI and NcoI, while the PCR product of the latter gene was cleaved with restriction enzymes NcoI and NheI. These two types of DNA fragments were cloned into pTN-B5R cleaved with restriction enzymes HpaI and NheI to construct pTN-B5Rgfp. In order to insert two repeats of a 22-base target sequence of let7a microRNA, two synthetic DNAs (5′-ctagcAACTATACAACCTACTACCTCAcgatAACTATACAACCTACTACCTCAc gcgta-3′ (SEQ ID NO: 77) and 5′-ccggtacgcgTGAGGTAGTAGGTTGTATAGTTatcgTGAGGTAGTAGGTTGTATA GTTg-3′ (SEQ ID NO: 78)) were annealed. This annealing product was cloned into pTN-B5R or pTN-B5Rgfp cleaved with restriction enzymes NheI and AgeI to construct pTN-B5R-let7ax2 or pTN-B5Rgfp-let7ax2. Similarly, for the insertion of two repeats of a 22-base mutated target sequence of let7a microRNA, two synthetic DNAs 5′-ctagcAATTACACGACTTATTATTTGAcgatAATTACACGACTTATTATTTGAcg cgta-3′ (SEQ ID NO: 79) and 5′-ccggtacgcgTCAAATAATAAGTCGTGTAATTatcgTCAAATAATAAGTCGTGTA ATTg-3′ (SEQ ID NO: 80) were used to construct pTN-B5R-let7a mutx2 or pTN-B5Rgfp-let7a mutx2. Furthermore, two synthetic DNAs (5′-cgcgtAACTATACAACCTACTACCTCAtcacAACTATACAACCTACTACCTCA-3′ (SEQ ID NO: 81) and 5′-ccggTGAGGTAGTAGGTTGTATAGTTgtgaTGAGGTAGTAGGTTGTATAGTTa-3′ (SEQ ID NO: 82)) were annealed, and the resulting DNA fragment was cloned into pTN-B5R-let7ax2 or pTN-B5Rgfp-let7ax2 cleaved with restriction enzymes MluI and AgeI to construct pTN-B5R-let7a or pTN-B5Rgfp-let7a having four repeats of the target sequence of let7a microRNA. Similarly, for the insertion of a mutated target sequence, two synthetic DNAs 5′-cgcgtAATTACACGACTTATTATTTGAtcacAATTACACGACTTATTATTTGA-3′ (SEQ ID NO: 83) and 5′-ccggTCAAATAATAAGTCGTGTAATTgtgaTCAAATAATAAGTCGTGTAATTa-3′ (SEQ ID NO: 84) were used to construct pTN-B5-let7a mut, or pTN-B5Rgfp-let7a mut.

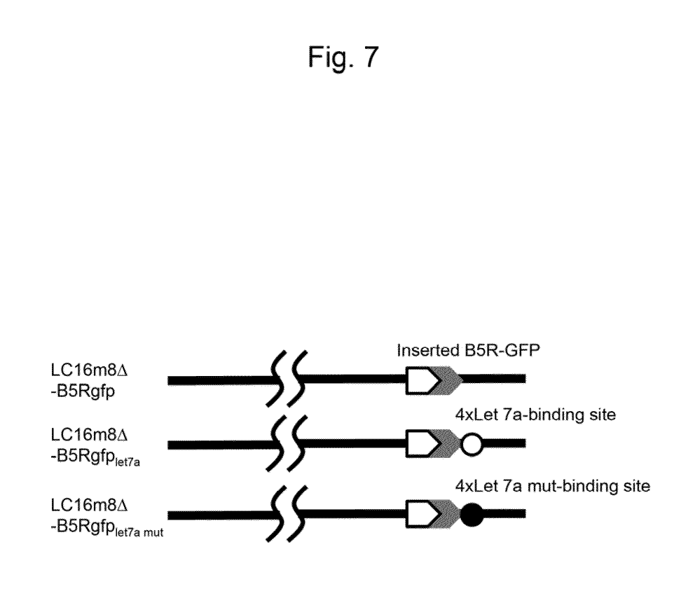

Each recombinant virus (FIG. 7) was prepared using the pTN-B5R, pTN-B5R-let7a or pTN-B5R-let7a mut, pTN-B5Rgfp, or pTN-B5Rgfp-let7a or pTN-B5Rgfp-let7a mut transfer vector in the same way as the method described in Reference Example 2. Plaques were purified with large plaques or EGFP expression as an index, followed by PCR and sequence confirmation by direct sequencing. Viral clones without problems in the nucleotide sequence were selected and cultured in large amounts in RK13 cells. Then, the virus titer was determined in the RK13 cells for the subsequent experiment.

### Example 2

### Establishment of Rapid and Convenient Evaluation System for MicroRNA-Controlled Proliferation-Type Vaccinia Virus

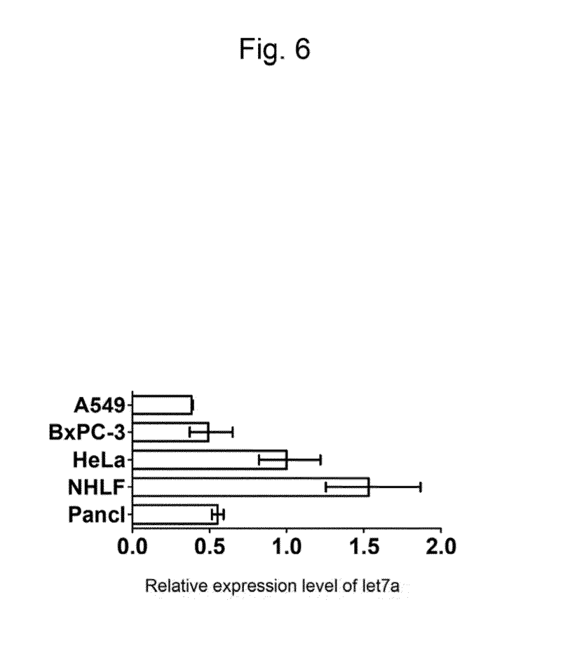

Total RNA containing small RNA was collected from human tumor cells using mirVana miRNA Isolation kit (Applied Biosystems, Inc.) according to the manual. Each tumor cell-derived let7a microRNA (Product ID; 000377) was quantified by the TaqMan method using 10 ng of each collected RNA and TaqMan (registered trademark) MicroRNA Assays (Applied Biosystems, Inc.) according to the manual. RNU6B (Product ID; 001093) was used as an internal control. The relative expression level of the let7a microRNA was calculated using the comparative Ct method based on HeLa cells. As a result, approximately 60% expression reduction for A549 cells, approximately 50% expression reduction for BxPC-3 cells, and approximately 45% expression reduction for Panel cells were observed with respect to the HeLa cells. By contrast, 1.5-fold expression with respect to the HeLa cells was observed in NHLF (normal human lung fibroblast) (FIG. 6).

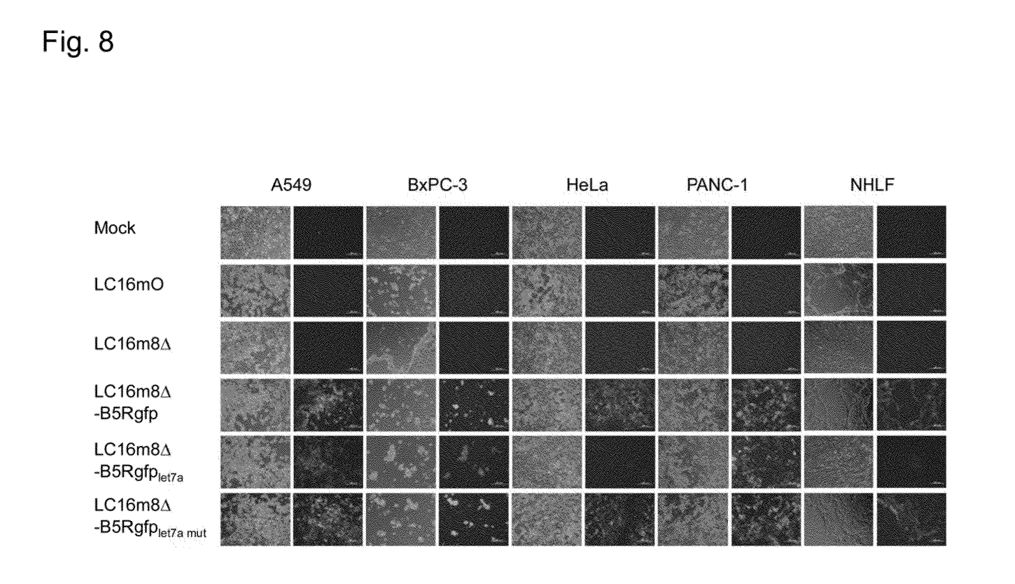

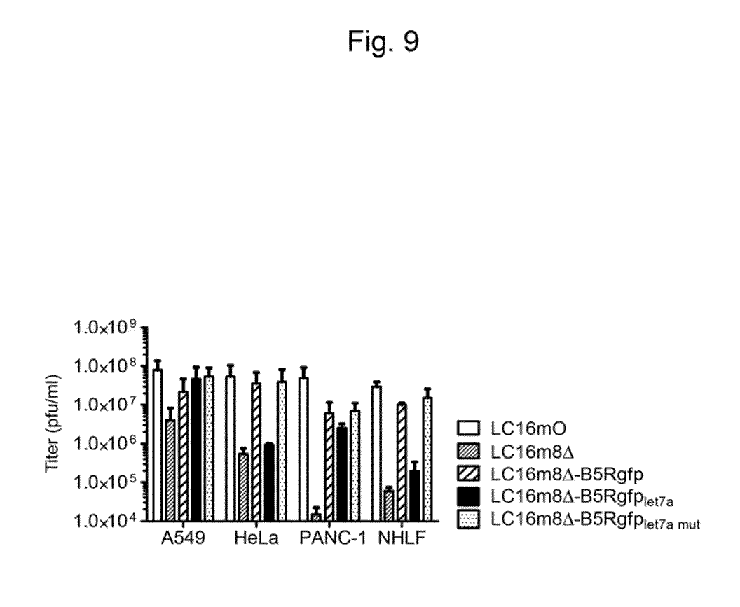

Each cancer cell line cultured in a 24-well dish was infected with each microRNA-controlled proliferation-type vaccinia virus having the viral genome shown in FIG. 7 at MOI=0.1 and cultured at 37° C. for 3 days. Then, these live cells were observed in the bright field and in a fluorescent manner using a fluorescence microscope (Olympus Corp.). As a result, all of the viruses having an insert of EGFP-fused B5R, EGFP-fused B5R with the let7a target sequence in 3′ UTR, or EGFP-fused B5R with the mutated let7a target sequence in 3′ UTR exhibited equivalent cytopathy in A549, BxPC-3, and Panel cells with low let7a microRNA expression. In addition, EGFP expression was confirmed in these degenerated cells. By contrast, neither cytopathy nor EGFP expression derived from LC16m8Δ-B5Rgfpₗₑₜ₇ₐ was observed in HeLa and NHLF cells with high let7a microRNA expression (FIG. 8). Furthermore, the infected cells of each line were recovered, frozen and thawed, and then sonicated. After centrifugation (2,000 rpm, min), the supernatant was recovered as a viral solution. The virus titer of each viral solution (1 ml) was determined in RK13 cells. As a result, LC16m8Δ-B5Rgfp, LC16m8Δ-B5Rgfpₗₑₜ₇ₐ, and LC16m8Δ-B5Rgfp_{let7a mut} proliferated in A549 and Panel cells at equivalent levels which were higher than that of LC16m8Δ and substantially comparable to the proliferation of LC16mO. By contrast, the proliferation of LC16m8Δ-B5Rgfpₗₑₜ₇ₐ in HeLa and NHLF cells was equivalent to that of LC16m8Δ and drastically reduced compared with the other viruses (FIG. 9). As is evident from these results, the microRNA-controlled virus exhibited cytopathy and efficient viral proliferation because B5R expression was repressed in cells expressing the microRNA but was achieved in cells with low expression of the microRNA, in synchronization with gene expression regulation based on the intracellular microRNA-based control mechanism. In addition, the expression of the B5R gene fused with the EGFP gene allows easy observation of B5R expression in live cells under a fluorescence microscope. This system is useful for the convenient and rapid evaluation of other viruses regulated by microRNAs other than let7a.

### Example 3

### Construction of MicroRNA-Controlled Proliferation-Type Vaccinia Virus Expressing Foreign Genes

In order to construct a recombinant virus expressing two types of foreign genes (firefly luciferase gene and EGFP gene) inserted in hemagglutinin (HA) gene, luciferase gene with the sequences of SfiI and FseI restriction enzymes added to both ends was amplified with a pGL4.20 plasmid (Promega Corp.) as a template using two primers 5′-GCGGCCGGACCGGCCACCATGGAAGATGCCAAAAA-3′ (SEQ ID NO: 85) and 5′-ATGGCCGGCCTTACACGGCGATCTTGCCGC-3′ (SEQ ID NO: 86). This PCR product was cleaved with restriction enzymes SfiI and FseI and cloned into the corresponding restriction site of pVNC110 (Suzuki H et al., Vaccine. 2009 11; 27 (7): 966-971) to construct pVNC110-Luc. An EGFP gene fragment obtained by the cleavage of the pEGFP-N1 plasmid with restriction enzymes SmaI and NotI was cloned into the corresponding restriction site of pIRES (Clontech Laboratories, Inc.) to construct pIRES-EGFP. Then, an IRES-EGFP gene fragment obtained by the cleavage of pIRES-EGFP with restriction enzymes MluI and NotI was blunted at both ends using T4 DNA polymerase. This blunt-ended gene fragment was cloned into the blunt-ended site of pVNC110-Luc treated with a restriction enzyme FseI to construct pVNC110-Luc/IRES/EGFP.

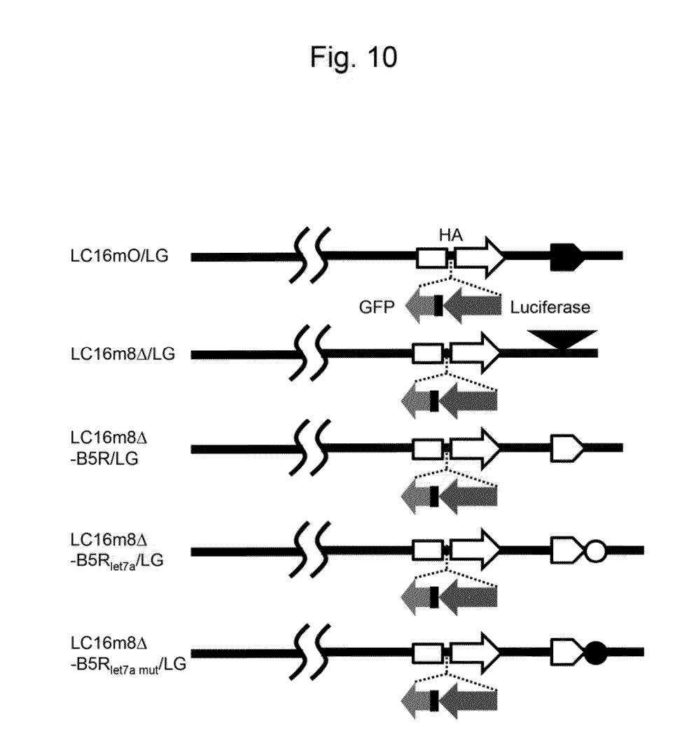

Each recombinant virus (FIG. 10) was prepared in the same way as the method described in Reference Example 2 by infecting RK13 cells with each vaccinia virus (LC16mO strain, LC16m8Δ, LC16m8Δ-B5R prepared in Reference Example 2, or LC16m8Δ-B5Rₗₑₜ₇ₐ or LC16m8Δ-B5R_{let7a mut} prepared in Example 1) and causing the cells to incorporate the transfer vector plasmid DNA pVNC110-Luc/IRES/EGFP. Plaques were purified with plaque size or EGFP expression as an index, followed by PCR and sequence confirmation by direct sequencing. Viral clones without problems in the nucleotide sequence were selected and cultured in large amounts in RK13 cells. Then, the virus titer was determined in the RK13 cells for the subsequent experiment.

### Example 4

### Properties of MicroRNA-Controlled Proliferation-Type Vaccinia Virus Expressing Foreign Genes

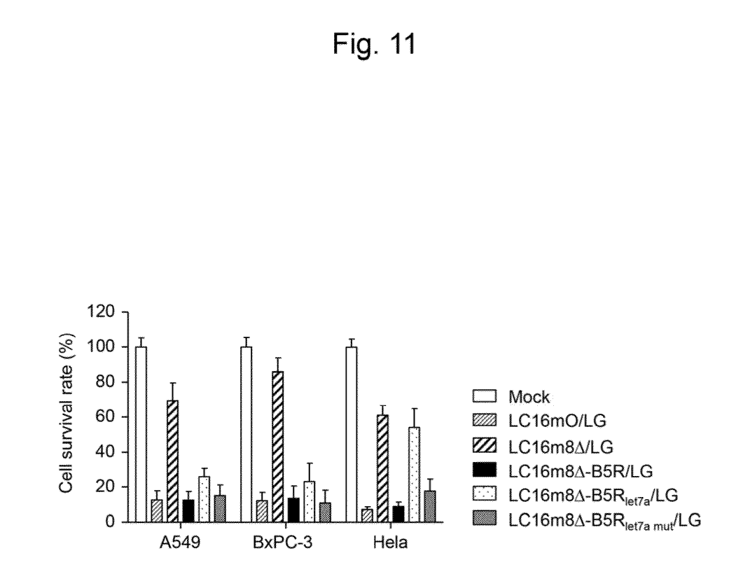

Each cancer cell line cultured in a 96-well dish was infected with each microRNA-controlled proliferation-type vaccinia virus having the viral genome shown in FIG. 10 at MOI=0.5 and cultured at 37° C. for 5 days. Then, the number of live cells was counted by the method described in Reference Example 2 (FIG. 11). As a result, the cell-killing effect of LC16m8Δ-B5Rₗₑₜ₇ₐ/LG having the let7a target sequence insert was equivalent to that of the LC16m8Δ lacking the B5R gene, in HeLa cells highly expressing let7a and was significantly reduced compared with LC16m8Δ-B5R/LG having the B5R insert or LC16m8Δ-B5R_{let7a mut}/LG having the mutated let7a target sequence insert. By contrast, its cell-killing effect was found to be equivalent to that of LC16m8Δ-B5R/LG or LC16m8Δ-B5R_{let7a mut}/LG and to be potent in A549 and BxPC-3 cells with low let7a expression. As is evident from these results, the microRNA-controlled proliferation-type vaccinia virus expressing the foreign genes also exhibited cell-killing effect based on efficient viral proliferation, because B5R expression was repressed in cells expressing the microRNA but was achieved in cells with low expression of the microRNA.

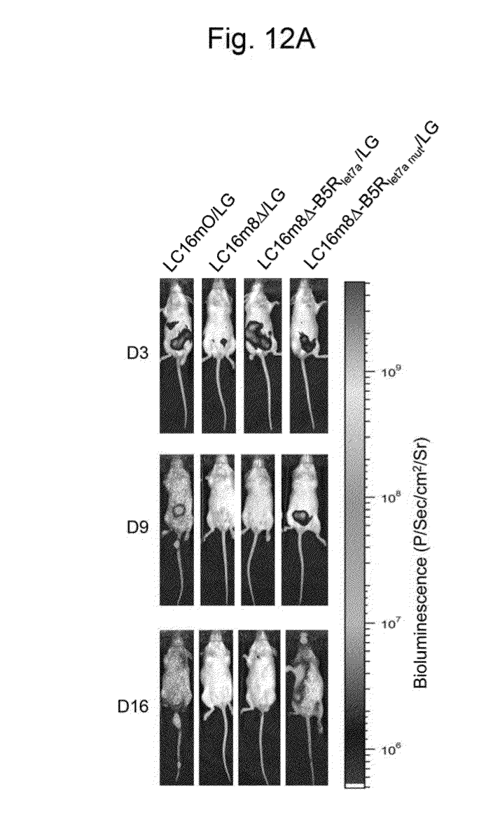

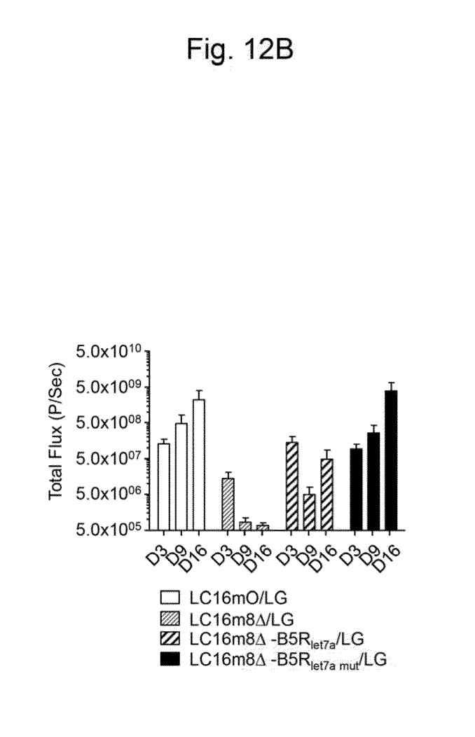

This microRNA-controlled virus was further examined for the presence or absence of its functions in mouse bodies. Since Let7a is highly expressed in all mouse normal tissues, LC16m8Δ-B5Rₗₑₜ₇ₐ/LG is presumed to fail to proliferate in mouse bodies. 10⁷ pfu of each luciferase-expressing virus was intraperitoneally administered to each SCID mouse (each group involving 3 individuals). As described in Reference Example 1, luciferin was administered thereto 3, 9, and 16 days later, and luciferase expression in cells with viral infection and proliferation was noninvasively monitored (FIG. 12A) and numerically converted (FIG. 12B). As a result, intraperitoneal expression was confirmed in all the virus-administered groups 3 days after the virus administration. This luciferase expression was numerically converted and statistically analyzed by two-way analysis of variance (two-way ANOVA). As a result, no significant difference was confirmed among all the viruses. By contrast, the proliferation of LC16m8Δ-B5Rₗₑₜ₇ₐ/LG was significantly reduced 9 and 16 days after the administration, as in LC16m8Δ/LG lacking the B5R gene, whereas the proliferation of LC16mO/LG and LC16m8Δ-B5R_{let7a mut}/LG was systemically spread over time without remaining only at the intraperitoneal administration site and was consistent with the developed pocks seen mainly in the tail, limbs, and oral cavity (FIG. 12A). As a result of similar statistical analysis, the proliferation of the virus LC16m8Δ-B5Rₗₑₜ₇ₐ/LG was confirmed 16 days after the administration to have a significant difference from LC16mO/LG and LC16m8Δ-B5R_{let7a mut}/LG, but no significant difference from LC16m8Δ (FIG. 12B). These results demonstrated that the proliferative capacity of the microRNA-controlled proliferation-type virus expressing the foreign genes was significantly reduced even in the normal tissues of immunodeficient SCID mice by let7a-based control.

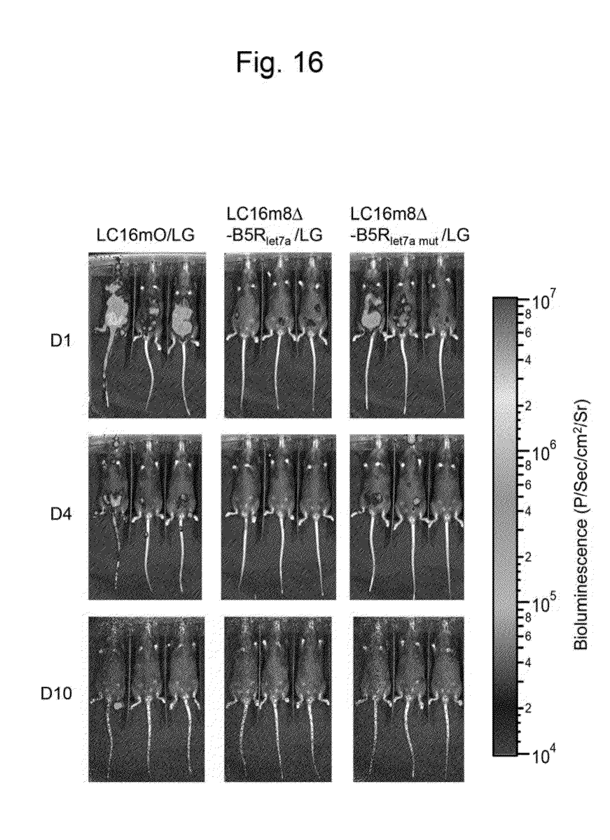

Next, 10⁸ pfu of each luciferase-expressing virus was intraperitoneally administered to each C57BL/6 mouse (each group involving 3 individuals). As described in Reference Example 1, luciferin was administered thereto 1, 4, and 10 days later, and luciferase expression in cells with viral infection and proliferation was noninvasively monitored (FIG. 16). As a result, the proliferation of LC16m8Δ-B5Rₗₑₜ₇ₐ/LG was low with slight intraperitoneal luciferase expression even 1 day after the virus administration, and was not confirmed 4 days thereafter. By contrast, the proliferation of LC16mO/LG and LC16m8Δ-B5R_{let7a mut}/LG was confirmed with strong intraperitoneal expression 1 day after the virus administration and was systemically (mainly in the tail, limbs, and oral cavity) spread 4 days thereafter without remaining only at the intraperitoneal administration site. However, the luciferase expression was also slight in the LC16mO/LG or LC16m8Δ-B5R_{let7a mut}/LG virus-administered group 10 days thereafter. These results demonstrated that the proliferative capacity of the microRNA-controlled proliferation-type virus expressing the foreign genes was very significantly reduced in the normal tissues of immunologically functional C57BL/6 mice within 24 hours after its infection by let7a-based control to eliminate the virus.

### Example 5

### Anticancer Effect and Safety of MicroRNA-Controlled Proliferation-Type Vaccinia Virus Expressing Foreign Gene

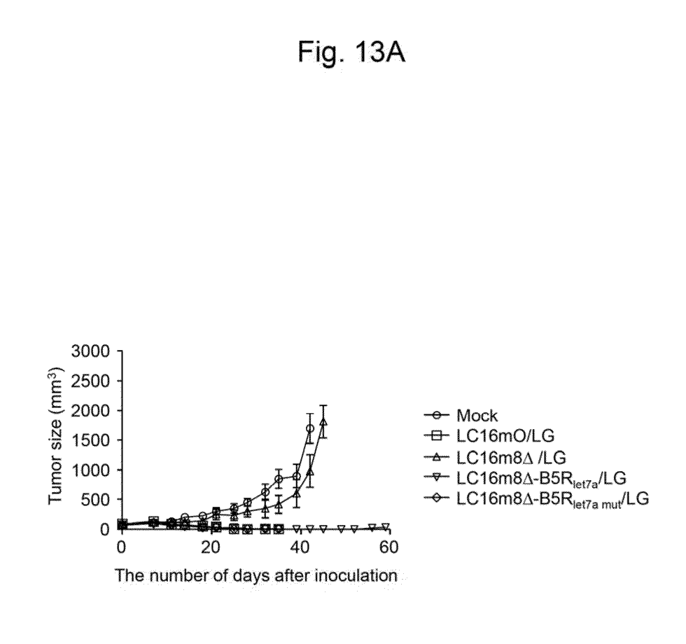

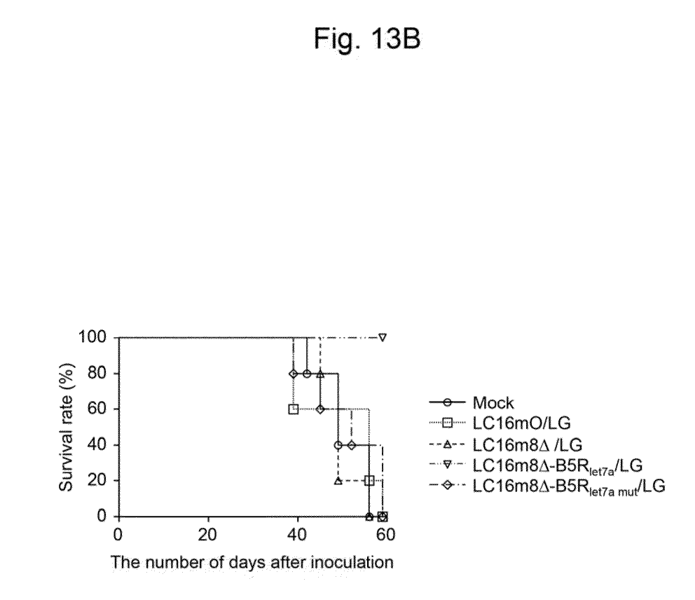

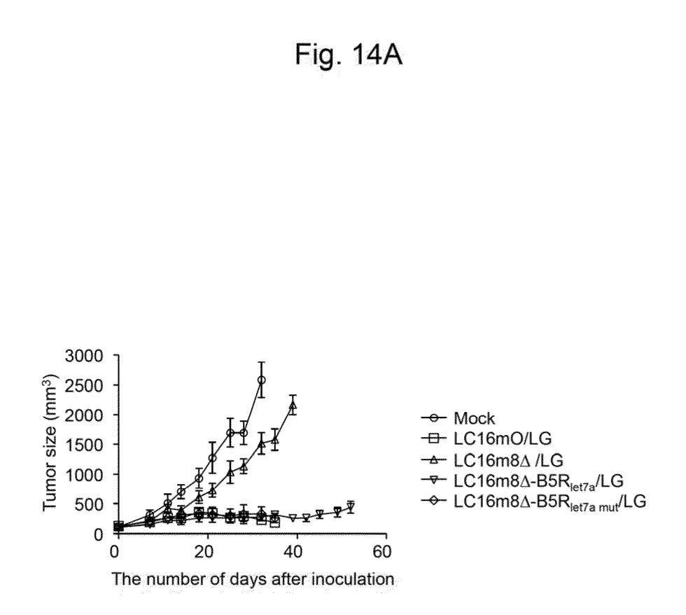

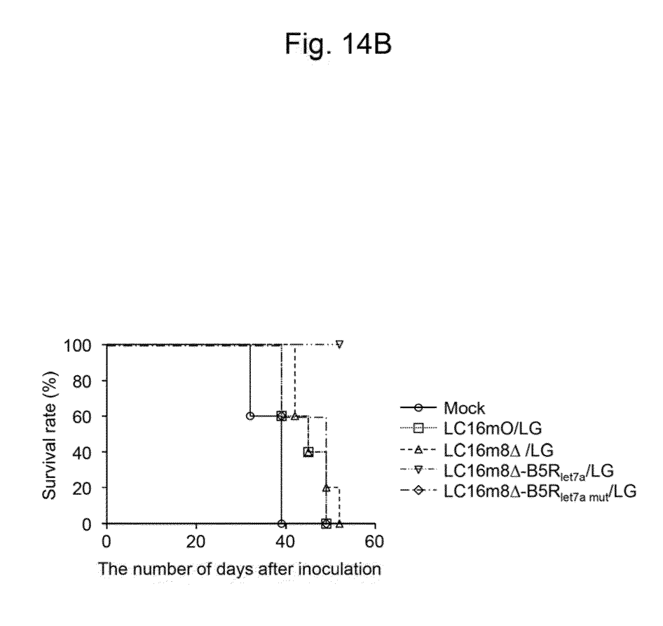

5×10⁶ BxPC-3 or A549 cells were subcutaneously transplanted to the right ventral region of each immunodeficient nude mouse. The point in time when the tumor mass reached approximately 100 mm³ (size calculated according to the equation V (tumor volume)=LW²/2 wherein the major axis L and the minor axis W of the tumor mass were measured via the skin using a vernier caliper) was defined as day 0. 10⁷ pfu of each virus was administered into the tumor at days 0, 3, and 6 (a total of three times) (each group involving 5 individuals). As a result, LC16mO/LG, LC16m8Δ-B5Rₗₑₜ₇ₐ/LG, or LC16m8Δ-B5R_{let7a mut}/LG exhibited potent anticancer effect on the mice bearing BxPC-3-derived cancer and was confirmed by two-way ANOVA statistical analysis to have a very significant difference in tumor volume 21 to 35 days later from a mock control group inoculated with no virus (FIG. 13A). The LC16m8Δ group was confirmed to very significantly differ in tumor volume 32 to 35 days later from the mock control group, but was euthanized up to 56 days after the treatment because the tumor volume reached 2500 mm³ in all the mice. By contrast, the LC16mO/LG or LC16m8Δ-B5R_{let7a mut}/LG-administered mice all died or were euthanized up to 59 days after the treatment due to rapid weight loss with pocks systemically developed. In contrast to these strains, the LC16m8Δ-B5Rₗₑₜ₇ₐ/LG-administered group was confirmed by the Log-rank test to have a very significant difference in survival rate from other groups. In this group, 100% mice survived 59 days after the treatment, and the complete tumor elimination was observed in four out of these five mice (FIG. 13B). Likewise, potent anticancer effect was also shown without adverse reactions in the LC16m8Δ-B5Rₗₑₜ₇ₐ/LG-administered group of the mice bearing A549-derived cancer, though the complete disappearance of the tumor was not observed in any of the mice (FIGS. 14A and 14B). All the virus-administered groups were confirmed by the Log-rank test to have a significant difference in survival rate from the mock control group. However, the LC16m8Δ group was euthanized up to 56 days after the treatment because the tumor volume reached 2500 mm³ in all the mice. All the mice in the LC16mO/LG and LC16m8Δ-B5R_{let7a mut}/LG groups died or were euthanized up to 49 days after the treatment due to rapid weight loss with pocks systemically developed.

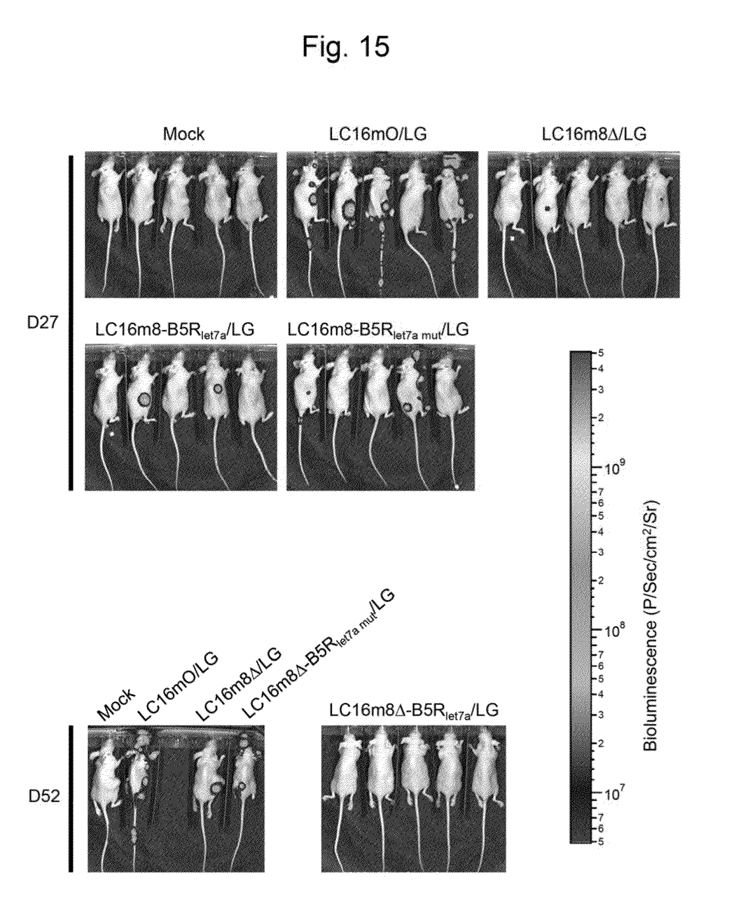

Next, luciferin was administered to the mice bearing BxPC-3-derived cancer 27 and 52 days after the treatment. Viral proliferation in the mouse bodies was noninvasively monitored as described in Example 4. As a result, viral proliferation was seen in the systemic normal tissues of the LC16mO/LG and LC16m8Δ-B5R_{let7a mut}/LG-administered mice 27 days after the treatment, and was increased 52 days thereafter and over time with rapid weight loss confirmed. By contrast, viral proliferation was restricted only to the transplanted tumor mass in the LC16m8Δ-B5Rₗₑₜ₇ₐ/LG-administered mice 27 days after the treatment, and was not seen in normal tissues of the mice (including mice whose tumor completely disappeared). This viral proliferation also disappeared 52 days thereafter in the mice whose tumor disappeared (FIG. 15).

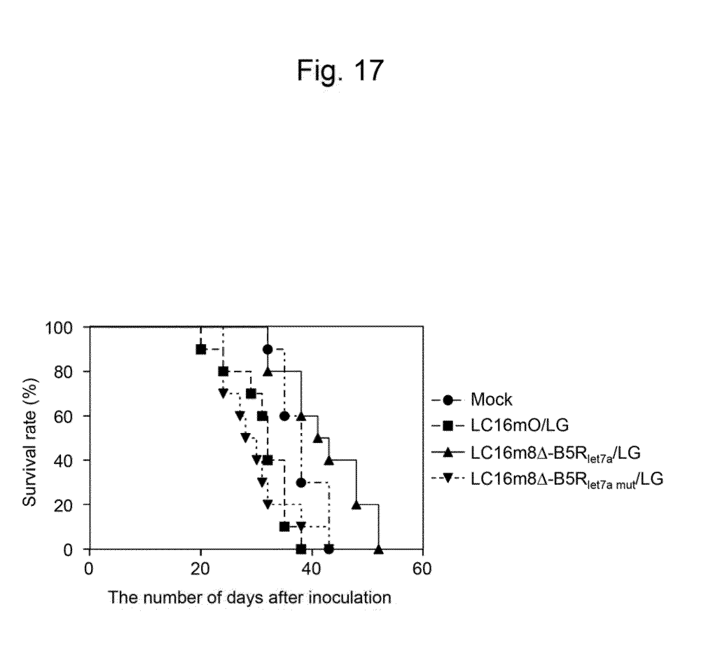

Meanwhile, BxPC-3 cells (5×10⁶ cells) were intraperitoneally administered to each SCID mouse. Seven days thereafter, 10⁷ pfu of each virus was intraperitoneally administered thereto (each group involving 10 individuals). As a result, the LC16mO/LG or LC16m8Δ-B5R_{let7a mut}/LG-administered mice all died or were euthanized due to rapid weight loss with pocks systemically developed up to 24 to 43 days after the treatment, earlier than death or euthanasia resulting from the tumors in the mice in a mock control group inoculated with no virus. By contrast, the LC16m8Δ-B5Rₗₑₜ₇ₐ/LG-administered mice were confirmed by the Log-rank test to have a very significant difference in survival rate from the LC16mO/LG or LC16m8Δ-B5R_{let7a mut}/LG-administered mice, but finally, all died or were euthanized due to substantially the same viral toxicity as in the LC16mO/LG or LC16m8Δ-B5R_{let7a mut}/LG-administered mice (FIG. 17).

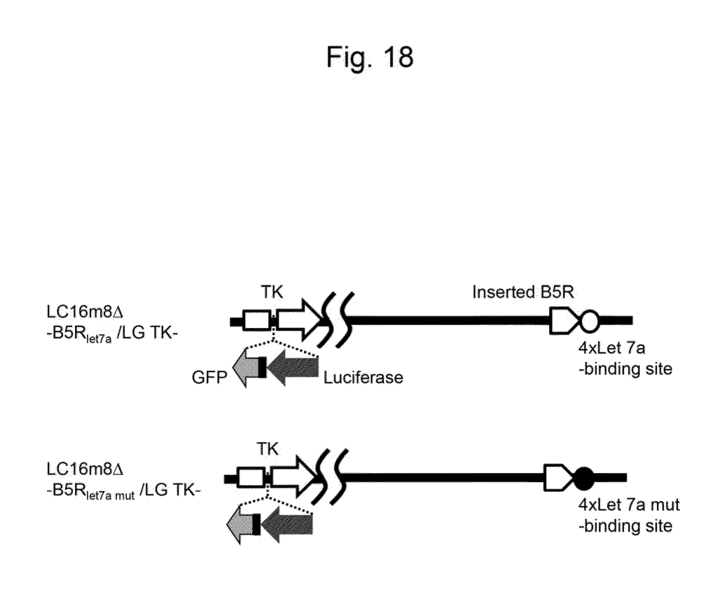

LC16m8Δ-B5Rₗₑₜ₇ₐ/LG has inserts of two types of foreign genes (firefly luciferase gene and EGFP gene) in hemagglutinin (HA) gene. Thus, two types of foreign genes were inserted to thymidine kinase (TK) gene to prepare a let7a-controlled recombinant virus LC16m8Δ-B5Rₗₑₜ₇ₐ/LG TK- lacking TK. First, the TK gene region was amplified with the genomic DNA of the LC16mO strain as a template using two primers 5′-cgCAGCTGAGCTTTTGCGATCAATAAATG-3′ (SEQ ID NO: 88) and 5′-TTCAGCTGAATATGAAGGAGCAA-3′ (SEQ ID NO: 89). This PCR product was cleaved with a restriction enzyme PvuII and cloned into the corresponding restriction site of a pUC19 vector to prepare pTK. Furthermore, two synthetic DNAs (5′-aattgcatgcgtcgacattaatGGCCGGACCGGCCttcgaag-3′ (SEQ ID NO: 90) and 5′-aattettcgaaGGCCGGTCCGGCCattaatgtcgacgcatgc-3′ (SEQ ID NO: 91)) were annealed. This annealing product was cloned into pTK cleaved with a restriction enzyme EcoRI to construct pTK-MSC. For the insertion of a synthetic vaccinia virus promoter (Hammond et al., Journal of Virological Methods. 1997 66: 135-138), two synthetic DNAs (5′-TCGAaattggatcagctattttttttattatggcatataaataaggtcgaGGTACCaaaaattgaaaaactattctaat ttattgcacGGCCGGAC-3′ (SEQ ID NO: 92) and 5′-CGGCCgtgcaataaattagaatagtttttcaatttttGGTACCtcgaccttatttatatgccaaaaaaaaaaaaaaaa aagctgatccaatt-3′ (SEQ ID NO: 93)) were annealed. This annealing product was cloned into pTK-MSC cleaved with restriction enzymes SfiI and SalI to construct pTK-SP-MSC. A Luc/IRES/EGFP gene fragment obtained by the cleavage of the pVNC110-Luc/IRES/EGFP plasmid with restriction enzymes SfiI and EcoRI was cloned into the corresponding restriction site of pTK-SP-MSC to construct pTK-SP-LG. Each recombinant virus (FIG. 18) was prepared according to a slight modification of the method described in Reference Example 2 by infecting 143 cells with each vaccinia virus (LC16m8Δ-B5Rₗₑₜ₇ₐ or LC16m8Δ-B5R_{let7a mut} prepared in Example 1) and causing the cells to incorporate the transfer vector plasmid DNA pTK-SP-LG in the presence of 25 μg/ml BUdR (bromodeoxyuridine). Plaques were purified with EGFP expression as an index, followed by PCR and sequence confirmation by direct sequencing. Viral clones without problems in the nucleotide sequence were selected and cultured in large amounts in RK13 cells. Then, the virus titer was determined in the RK13 cells for the subsequent experiment.

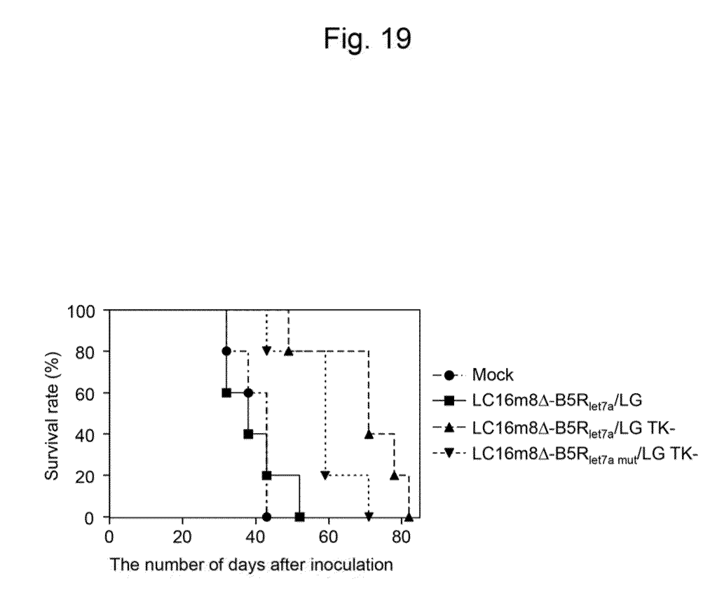

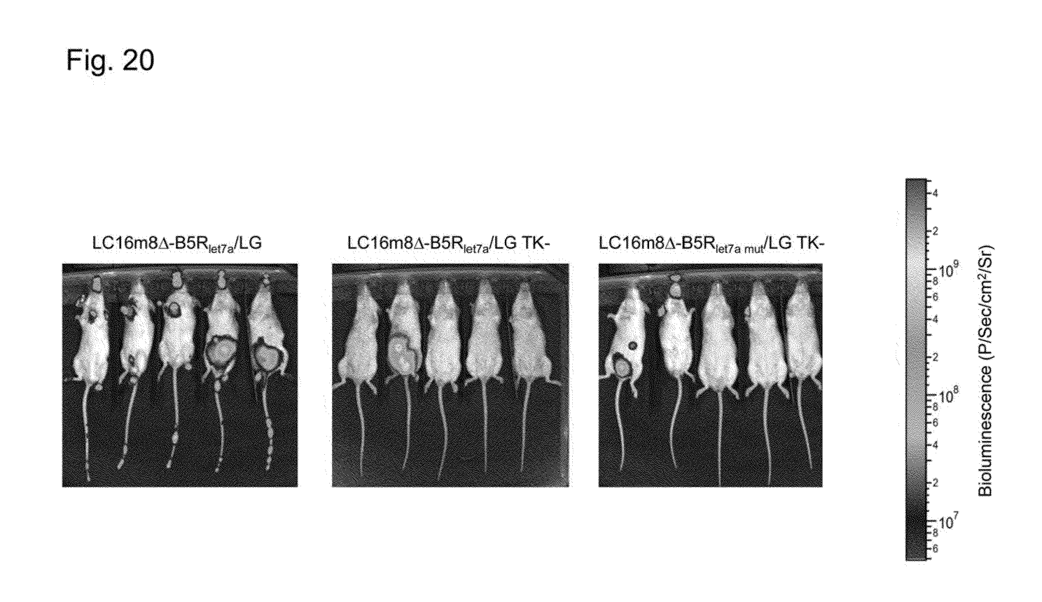

As described above, BxPC-3 cells (5×10⁶ cells) were intraperitoneally administered to each SCID mouse. Seven days thereafter, 10⁷ pfu of each virus was intraperitoneally administered thereto (each group involving 5 individuals). As a result, the LC16m8Δ-B5Rₗₑₜ₇ₐ/LG TK- group was confirmed by the Log-rank test to have a very significant difference in survival rate from the mice in a mock control group inoculated with no virus and the LC16m8Δ-B5Rₗₑₜ₇ₐ/LG-administered mice, and was free from observable adverse reactions attributed to viral toxicity (FIG. 19). Next, luciferin was administered 29 days later. Viral proliferation in the mouse bodies was noninvasively monitored as described in Example 4. As a result, viral proliferation was observed in the normal tissues of the LC16m8Δ-B5Rₗₑₜ₇ₐ/LG and LC16m8Δ-B5Rₗₑₜ₇ₐ₋ₘᵤₜ/LG TK- administered mice, but was restricted only to the intraperitoneal tumors in LC16m8Δ-B5Rₗₑₜ₇ₐ/LG TK- with no viral proliferation seen in normal tissues (FIG. 20). As is evident from these results, the insertion of foreign genes in the TK gene enhanced the therapeutic index of the microRNA-controlled proliferation-type vaccinia virus, compared with the insertion in the HA gene.

The results described above demonstrated that the microRNA-controlled proliferation-type vaccinia virus of the present invention was a virus having both of antitumor effect based on potent oncolytic effect and high safety in cancer-bearing mouse models.

### INDUSTRIAL APPLICABILITY

The miRNA-controlled vaccinia virus of the present invention can be used in cancer treatment.

### Free Text for Sequence Listing

### SEQ ID NOs: 65-76, 85, 86, 88, and 89: Primer

### SEQ ID NOs: 77-84 and 90-93: Synthetic

All publications, patents, and patent applications cited herein are incorporated herein by reference in their entirety.

1. A microRNA-controlled vaccinia virus, in which a target sequence of a microRNA less expressed in a cancer cell than in a normal cell is inserted in a 3′ untranslated region of B5R gene associated with viral proliferation in a vaccinia virus, wherein the microRNA-controlled vaccinia virus specifically proliferates in the cancer cell and has an oncolytic property that specifically destroys the cancer cell.

2. The microRNA-controlled vaccinia virus according to claim 1, wherein the microRNA expressed in the normal cell represses the expression of the B5R gene to reduce the proliferative capacity of the microRNA-controlled vaccinia virus in the normal cell.

3. The microRNA-controlled vaccinia virus according to claim 1 or 2, wherein the B5R gene into which the microRNA target sequence is inserted in its 3′ untranslated region is introduced into an attenuated vaccinia virus lacking a portion or the whole of its B5R gene.

4. The microRNA-controlled vaccinia virus according to claim 1 or 2, wherein the vaccinia virus is an LC16 strain or an LC16mO strain.

5. The microRNA-controlled vaccinia virus according to claim 3, wherein the vaccinia virus is an LC16m8 strain lacking a portion of its B5R gene or an m8Δ strain lacking the whole of its B5R gene.

6. The microRNA-controlled vaccinia virus according to claim 1, wherein the microRNA less expressed in a cancer cell than in a normal cell is selected from the group consisting of let-7a (SEQ ID NO: 1), let-7b (SEQ ID NO: 2), let-7c (SEQ ID NO: 3), let-7d (SEQ ID NO: 4), let-7e (SEQ ID NO: 5), let-7f (SEQ ID NO: 6), miR-9 (SEQ ID NO: 7), miR-15a (SEQ ID NO: 8), miR-16-1 (SEQ ID NO: 9), miR-21 (SEQ ID NO: 10), miR-20a (SEQ ID NO: 11), miR-26a (SEQ ID NO: 12), miR-27b (SEQ ID NO: 13), miR-29a (SEQ ID NO: 14), miR-29b (SEQ ID NO: 15), miR-29c (SEQ ID NO: 16), miR-30a (SEQ ID NO: 17), miR-30d (SEQ ID NO: 18), miR-32 (SEQ ID NO: 19), miR-33a (SEQ ID NO: 20), miR-34a (SEQ ID NO: 21), miR-92a (SEQ ID NO: 22), miR-95 (SEQ ID NO: 23), miR-101 (SEQ ID NO: 24), miR-122 (SEQ ID NO: 25), miR-124 (SEQ ID NO: 26), miR-125a (SEQ ID NO: 27), miR-125b (SEQ ID NO: 28), miR-126 (SEQ ID NO: 29), miR-127 (SEQ ID NO: 30), miR-128 (SEQ ID NO: 31), miR-133b (SEQ ID NO: 32), miR-139-5p (SEQ ID NO: 33), miR-140 (SEQ ID NO: 34), miR-141 (SEQ ID NO: 35), miR-142 (SEQ ID NO: 36), miR-143 (SEQ ID NO: 37), miR-144 (SEQ ID NO: 38), miR-145 (SEQ ID NO: 39), miR-155 (SEQ ID NO: 40), miR-181a (SEQ ID NO: 41), miR-181b (SEQ ID NO: 42), miR-181c (SEQ ID NO: 43), miR-192 (SEQ ID NO: 44), miR-195 (SEQ ID NO: 45), miR-198 (SEQ ID NO: 46), miR-199a (SEQ ID NO: 47), miR-199b-5p (SEQ ID NO: 48), miR-200a (SEQ ID NO: 49), miR-203 (SEQ ID NO: 50), miR-204 (SEQ ID NO: 51), miR-205 (SEQ ID NO: 52), miR-217 (SEQ ID NO: 53), miR-218 (SEQ ID NO: 54), miR-219-5p (SEQ ID NO: 55), miR-220a (SEQ ID NO: 56), miR-220b (SEQ ID NO: 57), miR-220c (SEQ ID NO: 58), miR-222 (SEQ ID NO: 59), miR-223 (SEQ ID NO: 60), miR-224 (SEQ ID NO: 61), miR-345 (SEQ ID NO: 62), and miR-375 (SEQ ID NO: 63).

7. The microRNA-controlled vaccinia virus according to claim 1, wherein the microRNA-controlled vaccinia virus is deficient in one or more gene(s) whose loss of function resulting from deletion of the gene(s) is compensated for in the cancer cell, but is not compensated for in the normal cell.

8. The microRNA-controlled vaccinia virus according to claim 7, wherein the microRNA-controlled vaccinia virus is deficient at least in a thymidine kinase gene.

9. The microRNA-controlled vaccinia virus according to claim 8, wherein the microRNA-controlled vaccinia virus is further deficient in a hemagglutinin (HA) gene.

10. The microRNA-controlled vaccinia virus according to claim 9, wherein the microRNA-controlled vaccinia virus is further deficient in an F fragment.

11. The microRNA-controlled vaccinia virus according to claim 8, wherein the microRNA-controlled vaccinia virus is further deficient in a VGF gene.

12. A pharmaceutical composition for cancer treatment, comprising a microRNA-controlled vaccinia virus according to claim 1.

13. A microRNA-controlled vaccinia virus vector comprising a foreign DNA introduced in a microRNA-controlled vaccinia virus according to claim 1.

14. The microRNA-controlled vaccinia virus vector according to claim 13, wherein the foreign DNA is a marker DNA, a therapeutic gene having cytotoxic effect or immunostimulating effect, or a DNA encoding a cancer, viral, bacterial, or protozoal antigen.

15. A pharmaceutical composition for cancer treatment or for use as a vaccine against a cancer, a virus, a bacterium, or a protozoan, comprising a microRNA-controlled vaccinia virus vector according to claim 13.

16. A method for evaluating the therapeutic effect of a microRNA-controlled vaccinia virus according to claim 1 on cancer in a cancer patient, comprising the steps of:
(i) contacting the microRNA-controlled vaccinia virus with a cancer cell and a normal cell collected from the cancer patient; and
(ii) assaying the proliferation of the microRNA-controlled vaccinia virus in the cancer cell and the normal cell, wherein the microRNA-controlled vaccinia virus is determined to have therapeutic effect on cancer when proliferating in the cancer cell and not proliferating in the normal cell.

17. The method according to claim 16, wherein the microRNA-controlled vaccinia virus has a fusion gene of a B5R gene and a marker gene, and the therapeutic effect on cancer is evaluated on the basis of marker expression.


SEQUENCE LISTING (93 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
agagguaguagguugcauaguu

SEQ ID NO: 5 - RNA, 22 nt, Homo sapiens
ugagguaggagguuguauaguu

SEQ ID NO: 6 - RNA, 22 nt, Homo sapiens
ugagguaguagauuguauaguu

SEQ ID NO: 7 - RNA, 23 nt, Homo sapiens
ucuuugguuaucuagcuguauga

SEQ ID NO: 8 - RNA, 22 nt, Homo sapiens
uagcagcacauaaugguuugug

SEQ ID NO: 9 - RNA, 22 nt, Homo sapiens
uagcagcacguaaauauuggcg

SEQ ID NO: 10 - RNA, 22 nt, Homo sapiens
uagcuuaucagacugauguuga

SEQ ID NO: 11 - RNA, 23 nt, Homo sapiens
uaaagugcuuauagugcagguag

SEQ ID NO: 12 - RNA, 22 nt, Homo sapiens
uucaaguaauccaggauaggcu

SEQ ID NO: 13 - RNA, 21 nt, Homo sapiens
uucacaguggcuaaguucugc

SEQ ID NO: 14 - RNA, 22 nt, Homo sapiens
uagcaccaucugaaaucgguua

SEQ ID NO: 15 - RNA, 23 nt, Homo sapiens
uagcaccauuugaaaucaguguu

SEQ ID NO: 16 - RNA, 22 nt, Homo sapiens
uagcaccauuugaaaucgguua

SEQ ID NO: 17 - RNA, 22 nt, Homo sapiens
uguaaacauccucgacuggaag

SEQ ID NO: 18 - RNA, 22 nt, Homo sapiens
uguaaacauccccgacuggaag

SEQ ID NO: 19 - RNA, 22 nt, Homo sapiens
uauugcacauuacuaaguugca

SEQ ID NO: 20 - RNA, 21 nt, Homo sapiens
gugcauuguaguugcauugca

SEQ ID NO: 21 - RNA, 22 nt, Homo sapiens
uggcagugucuuagcugguugu

SEQ ID NO: 22 - RNA, 22 nt, Homo sapiens
uauugcacuugucccggccugu

SEQ ID NO: 23 - RNA, 22 nt, Homo sapiens
uucaacggguauuuauugagca

SEQ ID NO: 24 - RNA, 21 nt, Homo sapiens
uacaguacugugauaacugaa

SEQ ID NO: 25 - RNA, 22 nt, Homo sapiens
uggagugugacaaugguguuug

SEQ ID NO: 26 - RNA, 20 nt, Homo sapiens
uaaggcacgcggugaaugcc

SEQ ID NO: 27 - RNA, 24 nt, Homo sapiens
ucccugagacccuuuaaccuguga

SEQ ID NO: 28 - RNA, 22 nt, Homo sapiens
ucccugagacccuaacuuguga

SEQ ID NO: 29 - RNA, 22 nt, Homo sapiens
ucguaccgugaguaauaaugcg

SEQ ID NO: 30 - RNA, 22 nt, Homo sapiens
cugaagcucagagggcucugau

SEQ ID NO: 31 - RNA, 21 nt, Homo sapiens
ucacagugaaccggucucuuu

SEQ ID NO: 32 - RNA, 22 nt, Homo sapiens
uuugguccccuucaaccagcua

SEQ ID NO: 33 - RNA, 22 nt, Homo sapiens
ucuacagugcacgugucuccag

SEQ ID NO: 34 - RNA, 22 nt, Homo sapiens
cagugguuuuacccuaugguag

SEQ ID NO: 35 - RNA, 22 nt, Homo sapiens
uaacacugucugguaaagaugg

SEQ ID NO: 36 - RNA, 21 nt, Homo sapiens
cauaaaguagaaagcacuacu

SEQ ID NO: 37 - RNA, 21 nt, Homo sapiens
ugagaugaagcacuguagcuc

SEQ ID NO: 38 - RNA, 20 nt, Homo sapiens
uacaguauagaugauguacu

SEQ ID NO: 39 - RNA, 23 nt, Homo sapiens
guccaguuuucccaggaaucccu

SEQ ID NO: 40 - RNA, 23 nt, Homo sapiens
uuaaugcuaaucgugauaggggu

SEQ ID NO: 41 - RNA, 23 nt, Homo sapiens
aacauucaacgcugucggugagu

SEQ ID NO: 42 - RNA, 23 nt, Homo sapiens
aacauucauugcugucggugggu

SEQ ID NO: 43 - RNA, 22 nt, Homo sapiens
aacauucaaccugucggugagu

SEQ ID NO: 44 - RNA, 21 nt, Homo sapiens
cugaccuaugaauugacagcc

SEQ ID NO: 45 - RNA, 21 nt, Homo sapiens
uagcagcacagaaauauuggc

SEQ ID NO: 46 - RNA, 22 nt, Homo sapiens
gguccagaggggagauagguuc

SEQ ID NO: 47 - RNA, 23 nt, Homo sapiens
cccaguguucagacuaccuguuc

SEQ ID NO: 48 - RNA, 23 nt, Homo sapiens
cccaguguuuagacuaucuguuc

SEQ ID NO: 49 - RNA, 22 nt, Homo sapiens
uaacacugucugguaacgaugu

SEQ ID NO: 50 - RNA, 22 nt, Homo sapiens
gugaaauguuuaggaccacuag

SEQ ID NO: 51 - RNA, 22 nt, Homo sapiens
uucccuuugucauccuaugccu

SEQ ID NO: 52 - RNA, 22 nt, Homo sapiens
uccuucauuccaccggagucug

SEQ ID NO: 53 - RNA, 23 nt, Homo sapiens
uacugcaucaggaacugauugga

SEQ ID NO: 54 - RNA, 21 nt, Homo sapiens
uugugcuugaucuaaccaugu

SEQ ID NO: 55 - RNA, 21 nt, Homo sapiens
ugauuguccaaacgcaauucu

SEQ ID NO: 56 - RNA, 21 nt, Homo sapiens
ccacaccguaucugacacuuu

SEQ ID NO: 57 - RNA, 21 nt, Homo sapiens
ccaccaccgugucugacacuu

SEQ ID NO: 58 - RNA, 22 nt, Homo sapiens
acacagggcuguugugaagacu

SEQ ID NO: 59 - RNA, 21 nt, Homo sapiens
agcuacaucuggcuacugggu

SEQ ID NO: 60 - RNA, 22 nt, Homo sapiens
ugucaguuugucaaauacccca

SEQ ID NO: 61 - RNA, 21 nt, Homo sapiens
caagucacuagugguuccguu

SEQ ID NO: 62 - RNA, 22 nt, Homo sapiens
gcugacuccuaguccagggcuc

SEQ ID NO: 63 - RNA, 22 nt, Homo sapiens
uuuguucguucggcucgcguga

SEQ ID NO: 64 - DNA, 22 bp, Artificial
  Synthetic
aactatacaacctactacctca

SEQ ID NO: 65 - DNA, 21 bp, Artificial
  Primer
tcggaagcagtcgcaaacaac

SEQ ID NO: 66 - DNA, 22 bp, Artificial
  Primer
ataccatcgtcgttaaaagcgc

SEQ ID NO: 67 - DNA, 20 bp, Artificial
  Primer
cgtataatacgttggtctat

SEQ ID NO: 68 - DNA, 20 bp, Artificial
  Primer
gatcgtgccaatagtagtta

SEQ ID NO: 69 - DNA, 18 bp, Artificial
  Primer
caaactctcgaaagacgt

SEQ ID NO: 70 - DNA, 35 bp, Artificial
  Primer
gcaccggtgctagcttacggtagcaatttatggaa

SEQ ID NO: 71 - DNA, 39 bp, Artificial
  Primer
ccgctagcaccggtatataaatccgttaaaataattaat

SEQ ID NO: 72 - DNA, 17 bp, Artificial
  Primer
caggaaacagctatgac

SEQ ID NO: 73 - DNA, 22 bp, Artificial
  Primer
caaaatattttcgttgcgaaga

SEQ ID NO: 74 - DNA, 26 bp, Artificial
  Primer
caccatgggtagcaatttatggaact

SEQ ID NO: 75 - DNA, 35 bp, Artificial
  Primer
gcggccggaccggccaccatggtgagcaagggcga

SEQ ID NO: 76 - DNA, 28 bp, Artificial
  Primer
gcgctagcttacttgtacagctcgtcca

SEQ ID NO: 77 - DNA, 59 bp, Artificial
  Synthetic
ctagcaactatacaacctactacctcacgataactatacaacctactacctcacgcgta

SEQ ID NO: 78 - DNA, 59 bp, Artificial
  Synthetic
ccggtacgcgtgaggtagtaggttgtatagttatcgtgaggtagtaggttgtatagttg

SEQ ID NO: 79 - DNA, 59 bp, Artificial
  Synthetic
ctagcaattacacgacttattatttgacgataattacacgacttattatttgacgcgta

SEQ ID NO: 80 - DNA, 59 bp, Artificial
  Synthetic
ccggtacgcgtcaaataataagtcgtgtaattatcgtcaaataataagtcgtgtaattg

SEQ ID NO: 81 - DNA, 53 bp, Artificial
  Synthetic
cgcgtaactatacaacctactacctcatcacaactatacaacctactacctca

SEQ ID NO: 82 - DNA, 53 bp, Artificial
  Synthetic
ccggtgaggtagtaggttgtatagttgtgatgaggtagtaggttgtatagtta

SEQ ID NO: 83 - DNA, 53 bp, Artificial
  Synthetic
cgcgtaattacacgacttattatttgatcacaattacacgacttattatttga

SEQ ID NO: 84 - DNA, 53 bp, Artificial
  Synthetic
ccggtcaaataataagtcgtgtaattgtgatcaaataataagtcgtgtaatta

SEQ ID NO: 85 - DNA, 35 bp, Artificial
  Primer
gcggccggaccggccaccatggaagatgccaaaaa

SEQ ID NO: 86 - DNA, 30 bp, Artificial
  Primer
atggccggccttacacggcgatcttgccgc

SEQ ID NO: 87 - DNA, 3336 bp, Vaccinia virus
atggatttttttaaaaaggaaatacttgactggagtatatatttatctcttcattatatagcacgcgtgttttccaatttttccacatcccatataatacaggattataatctcgttcgaacatacgagaaagtggataaaacaatagttgattttttatctaggttgccaaatttattccatattttagaatatggggaaaatattctacatatttattctatggatgatgctaatacgaatattataattttttttctagatagagtattaaatattaataagaacgggtcatttatacacaatctcgggttatcatcatccattaatataaaagaatatgtatatcaattagttaataatgatcatccagataataggataagactaatgcttgaaaatggacgtagaacaagacattttttgtcctatatatcagatacagttaatatctatatatgtattttaataaatcatggattttatatagatgccgaagacagttacggttgtacattattacatagatgtatatatcactataagaaatcagaatcagaatcatacaatgaattaattaagatattgttaaataatggatccgatgtagataaaaaagatacgtacggaaacacaccttttatcctattatgtaaacacgatatcaacaacgtggaattgtttgagatatgtttagagaatgctaatatagactctgtagactttaatagatatacacctcttcattatgtctcatgtcgtaataaatatgattttgtaaagttattaatttctaaaggagcaaatgttaatgcgcgtaatagattcggaactactccattttattgtggaattatacacggtatctcgcttataaaactatatttggaatcagacacagagttagaaatagataatgaacatatagttcgtcatttaataatttttgatgctgttgaatctttagattatctattatccagaggagttattgatattaactatcgtactatatacaacgaaacatctatttacgacgctgtcagttataatgcgtataatacgttggtctatctattaaacaaaaatggtgattttgagacgattactactagtggatgtacatgtatttcggaagcagtcgcaaacaacaacaaaataataatggaagtactattgtctaaacgaccatctttgaaaattatgatacagtctatgatagcaattactaaacataaacagcataatgcagatttattgaaaatgtgtataaaatatactgcgtgtatgaccgattatgatactcttatagatgtacagtcgctacagcaatataaatggtatattttaagatgtttcgatgaaatagatatcatgaagagatgttatataaaaaataaaactgtattccaattagttttttgtatcaaagacattaatactttaatgagatacggtaaacatccttctttcgtgaagtgcactagtctcgacgtatacggaagtcgtgtacgtaatatcatagcatctattagatatcgtcagagattaattagtctattatccaagaagctggatgcgggagataaatggtcgtgttttcctaacgaaataaaatataaaatattggaaaactttaacgataacgaactatccacatatctaaaaatcttataaacattattaaaatataaaatctaagtggataaaatcacactacatcattgtttccttttagtgctcgacagtgtatactatttttaacgctcataaataaaaatgaaaacgatttccgttgttacgttgttatgcgtactacctgctgttgtttattcaacatgtactgtacccactatgaataacgctaaattaacgtctaccgaaacatcgtttaatgataaacagaaagttacatttacatgtgatcagggatatcattctttggatccaaatgctgtctgtgaaacagataaatggaaatacgaaaatccatgcaagaaaatgtgcacagtttctgattatgtctctgaattatatgataagccattatacgaagtgaattccaccatgacactaagttgcaacggcgaaacaaaatattttcgttgcgaagaaaaaaatggaaatacttcttggaatgatactgttacgtgtcctaatgcggaatgtcaacctcttcaattagaacacggatcgtgtcaaccagttaaagaaaaatactcatttggggaatatatgactatcaactgtgatgttggatatgaggttattggtgcttcgtacataagttgtacagctaattcttggaatgttattccatcatgtcaacaaaaatgtgatatgccgtctctatctaacggattaatttccggatctacattttctatcggtggcgttatacatcttagttgtaaaagtggttttacactaacggggtctccatcatccacatgtatcgacggtaaatggaatcccatactcccaacatgtgtacgatctaacgaaaaatttgatccagtggatgatggtcccgacgatgagacagatttgagcaaactctcgaaagacgttgtacaatatgaacaagaaatagaatcgttagaagcaacttatcatataatcatagtggcgttaacaattatgggcgtcatatttttaatctccgttatagtattagtttgttcctgtgacaaaaataatgaccaatataagttccataaattgctaccgtaaatataaatccgttaaaataattaataattaataacgaacaagtatcaaaagattaaagacttatagctagaatcaattgagatgtcttcttcagtggatgttgatatctacgatgccgttagagcatttttactcaggcactattataacaagagatttattgtgtatggaagaagtaacgccatattacataatatatacaggctatttacaagatgcgccgttataccgttcgatgatatagtacgtactatgccaaatgaatcacgtgttaaacaatgggtgatggatacacttaatggtataatgatgaatgaacgcgatgtttctgtaagcgttggcaccggaatactattcatggaaatgtttttcgattacaataaaaatagtatcaacaatcaactaatgtatgatataattaatagcgtatctataattctagctaatgagagatatagaagcgcttttaacgacgatggtatatacatccgtagaaatatgattaacaagttgtacggatacgcatctctaactactattggcacgatcgctggaggtgtttgttattatctgttgatgcatctagttagtttgtataaataa

SEQ ID NO: 88 - DNA, 29 bp, Artificial
  Primer
cgcagctgagcttttgcgatcaataaatg

SEQ ID NO: 89 - DNA, 23 bp, Artificial
  Primer
ttcagctgaatatgaaggagcaa

SEQ ID NO: 90 - DNA, 42 bp, Artificial
  Synthetic
aattgcatgcgtcgacattaatggccggaccggccttcgaag

SEQ ID NO: 91 - DNA, 42 bp, Artificial
  Synthetic
aattcttcgaaggccggtccggccattaatgtcgacgcatgc

SEQ ID NO: 92 - DNA, 99 bp, Artificial
  Synthetic
tcgaaattggatcagcttttttttttttttttttggcatataaataaggtcgaggtaccaaaaattgaaaaactattctaatttattgcacggccggac

SEQ ID NO: 93 - DNA, 92 bp, Artificial
  Synthetic
cggccgtgcaataaattagaatagtttttcaatttttggtacctcgaccttatttatatgccaaaaaaaaaaaaaaaaaagctgatccaatt